SEQUENCE LISTING (59 sequences)

SEQ ID NO: 1 - protein, 34 aa, human herpesvirus 1
DAATATRGRSAASRPTERPRAPARSASAPAAPVG

SEQ ID NO: 2 - protein, 16 aa, Drosophila sp.
RQIKIWFQNRRMKWKK

SEQ ID NO: 3 - protein, 13 aa, Human immunodeficiency virus type 1
GRKKRRQRRRPPQ

SEQ ID NO: 4 - protein, 13 aa, Human immunodeficiency virus type 1
GRKKRRQRRRPPQ

SEQ ID NO: 5 - protein, 13 aa, Human immunodeficiency virus type 1
GRRRRRRRRRPPQ

SEQ ID NO: 6 - protein, 12 aa, Homo sapiens
SQMTRQARRLYV

SEQ ID NO: 7 - protein, 18 aa, Human immunodeficiency virus type 1
TRQARRNRRRRWRERRQR

SEQ ID NO: 8 - protein, 15 aa, flock house virus
RRRRNRTRRNRRRVR

SEQ ID NO: 9 - protein, 19 aa, Unknown
  BMV Gag residues 7-25 cell pentetrating domain
KMTRAQRRAAARKNKRNTR

SEQ ID NO: 10 - protein, 13 aa, Human T-cell lymphotropic virus type 2
TRRQRTRRARRNR

SEQ ID NO: 11 - protein, 19 aa, Cowpea chlorotic mottle virus
KLTRAQRRAAARKNKRNTR

SEQ ID NO: 12 - protein, 17 aa, Unknown
  P22 N residues 14-30 cell penetrating domain
NAKTRRHERRRKLAIER

SEQ ID NO: 13 - protein, 27 aa, Homo sapiens
KRRIRRERNKMAAAKSRNRRRELTDDT

SEQ ID NO: 14 - protein, 28 aa, Homo sapiens
RIKAERKRMRNRIAASKSRKRKLERIAR

SEQ ID NO: 15 - protein, 22 aa, Unknown
  GCN4 residues 231-252 cell penetrating domain
KRARNTEAARRSRARKLQRMQK

SEQ ID NO: 16 - protein, 12 aa, Unknown
  PTD-4 cell penetrating domain
PIRRRKKLRRLK

SEQ ID NO: 17 - protein, 12 aa, Unknown
  PTD-5 cell penetrating domain
RRQRRTSKLMKR

SEQ ID NO: 18 - protein, 13 aa, Unknown
  Penetratin cell penetrating domain
GRKKRRQRRRPPQ

SEQ ID NO: 19 - protein, 26 aa, Unknown
  Transportan cell penetrating domain
GWTLNSAGYLLKINLKALAALAALIL

SEQ ID NO: 20 - protein, 18 aa, Unknown
  Amphipathic peptide cell penetrating domain
KLALKLALKALKAALKLA

SEQ ID NO: 21 - protein, 11 aa, Human immunodeficiency virus type 1
YGRKKRRQRRR

SEQ ID NO: 22 - protein, 25 aa, Unknown
  CR1 transmembrane peptide domain
AALLLAAAAAAAAAAAAAAAAAAAW

SEQ ID NO: 23 - protein, 30 aa, Unknown
  P24 transmembrane domain dequence
KKGLLLLLLLLLLLLLLLLLLLLLLLLKKA

SEQ ID NO: 24 - protein, 24 aa, Human immunodeficiency virus type 1
IAIVALVVAIIIAIVVWSIVIIEY

SEQ ID NO: 25 - protein, 24 aa, mammalian
LLFVITLPFWAVDAVANWYFGNDD

SEQ ID NO: 26 - protein, 26 aa, Bovine papillomavirus type 5
YVLFFLLLFLLLLLQMAAVLGLFLLF

SEQ ID NO: 27 - protein, 22 aa, Homo sapiens
IATGMVALLLLLVVALGIGLFM

SEQ ID NO: 28 - protein, 1 aa, Artificial Sequence
  Polylysine sequence having one or more lysine residues-Adapter domain for nucleic acid binding
K

SEQ ID NO: 29 - protein, 1 aa, Artificial Sequence
  HoK, histidylated polylysine residue having one or more lysines-Adapter domain for nucleic acid binding
K

SEQ ID NO: 30 - protein, 24 aa, Artificial Sequence
  4 sub 6 adapter domain for nucleic acid binding
LARLLARLLARLLARLLARLLARL

SEQ ID NO: 31 - protein, 12 aa, Artificial Sequence
  Branched peptide adapter domain for nucleic acid binding- (Lys-Lys-Lys-Lys) sub 2 Lys-Gly-Gly-Cys)
KKKKKKKKKGGC

SEQ ID NO: 32 - protein, 12 aa, Artificial Sequence
  Branched peptide adapter domain for nucleic acid binding- (Arg-Arg-Arg-Arg) sub 2 (Lys-Gly-Gly-Cys)
RRRRRWRRKGGC

SEQ ID NO: 33 - protein, 12 aa, Artificial Sequence
  Branched peptide adapter doamin for nucleic acid binding- (Arg-Trp-Arg-Arg) sub 2 Lys-Gly-Gly-Cys
RWRRRWRRKGGC

SEQ ID NO: 34 - protein, 8 aa, Artificial Sequence
  Oligoslysine with from 8 to 24 lysine residues
KKKKKKKK

SEQ ID NO: 35 - protein, 1 aa, Artificial Sequence
  polyarginine adapter domain for nucleic acid binding with two or more Arg residues
R

SEQ ID NO: 36 - protein, 35 aa, Human immunodeficiency virus type 1
GGGGYGRKKRRQRRRGGGGKKKKKKKKKKKKKKKK

SEQ ID NO: 37 - protein, 13 aa, Human immunodeficiency virus type 1
GGGGYGRKKRRQR

SEQ ID NO: 38 - protein, 50 aa, Human immunodeficiency virus type 1
GGGYGRKKRRQRRRGGGAALLLAAAAAAAAAAAAAAAAAAAWKKKKKKKK

SEQ ID NO: 39 - protein, 62 aa, Human immunodeficiency virus type 1
GGGGYGRKKRRQRRRGGGMPNLWFLLFLGLVAAMQLLLLLFLLLFFLVYWDHFECSCTGLPF

SEQ ID NO: 40 - DNA, 19 bp, Artificial Sequence
  bipolaroligonucleotide with the last T being 3′-dTn-5“
tatatgatggtaccgcagt

SEQ ID NO: 41 - protein, 35 aa, Human immunodeficiency virus type 1; identical to SEQ ID NO: 36

SEQ ID NO: 42 - protein, 13 aa, Human immunodeficiency virus type 1
GGGGYGRKKRRQR

SEQ ID NO: 43 - protein, 17 aa, Human immunodeficiency virus type 1
GGGYGRKKRRQRRRGGG

SEQ ID NO: 44 - protein, 20 aa, Unknown
  Intracellular domain of CR2
GGGGYGRKKRRQRRRGGGMP

SEQ ID NO: 45 - protein, 28 aa, Unknown
  Transmembrane domain of CR2
LWFLLFLGLVAAMQLLLLLFLLLFFLVY

SEQ ID NO: 46 - protein, 13 aa, Unknown
  Extracellular domain of CR2
WDHFECSCTGLPF

SEQ ID NO: 47 - protein, 20 aa, Unknown
  FITC-0-24 oligonucleotide cell membrane translocation facilitating peptide
GGGGKKKKKKKKKKKKKKKK

SEQ ID NO: 48 - protein, 15 aa, Human immunodeficiency virus type 1
GGGGYGRKKRRQRRR

SEQ ID NO: 49 - protein, 27 aa, Human immunodeficiency virus type 1
GGGGYGRKKRRQRRRGGGGKKKKKKKK

SEQ ID NO: 50 - protein, 31 aa, Human immunodeficiency virus type 1
GGGGYGRKKRRQRRRGGGGKKKKKKKKKKKK

SEQ ID NO: 51 - protein, 50 aa, Human immunodeficiency virus type 1; identical to SEQ ID NO: 38

SEQ ID NO: 52 - DNA, 24 bp, Artificial Sequence
  oligonucleotide translocation test sequence with a 3′ 3-hydroxy-1-propyl
tcagaactcacctgttagacgcca

SEQ ID NO: 53 - DNA, 83 bp, Artificial Sequence
  oligonucleotide translocation test sequence
ttgataagaggtcatttttgcggatggcttagagcttaattgctgaatctggtgctgtagctcaacatgttttaaatatgcaa

SEQ ID NO: 54 - DNA, 22 bp, Artificial Sequence
  oligonucleotide translocation test sequence
ccagactgagtatctcctatca

SEQ ID NO: 55 - protein, 2 aa, Artificial Sequence
  [(R)p(X)m]n-Mixed polyarginine copolymer where X is any amino acid, p, m and n equal any number
RX

SEQ ID NO: 56 - protein, 2 aa, Artificial Sequence
  [(K)p(X)m]n-mixed polylysine coploymer where X is any amino acid,p, m, and n equal any number
KX

SEQ ID NO: 57 - protein, 39 aa, Artificial Sequence
  Delta-CR1, fluorobiotinylated delta CR-1 Sequence
GGGGGGAALLLAAAAAAAAAAAAAAAAAAAWKKKKKKKK

SEQ ID NO: 58 - DNA, 12 bp, Artificial Sequence
  The listed sequence with a 3′ propylamine
aggaaaaaaaaa

SEQ ID NO: 59 - DNA, 30 bp, Artificial Sequence
  The listed sequence with a 3′ propylamine
aggaaggggggggtggtgggggagggggag

(19) United States
(12) Patent Application Publication
(10) Pub. No.: US 2003/0170826 A1
(43) Pub. Date: Sep. 11, 2003

(54) Peptides for facilitating composite receptor expression and translocation of macromolecules

(76) Inventors: Peter Rabinovich, Madison, CT (US); Patricia Bray-Ward, Madison, CT (US); David C. Ward, Madison, CT (US)

Correspondence Address: MORGAN, LEWIS & BOCKIUS LLP, 1701 MARKET STREET, PHILADELPHIA, PA, 19103-2921, US

(21) Appl. No.: 10/061,607

(22) Filed: Feb. 1, 2002

Related U.S. Application Data
(60) Provisional application No. 60/265,624, filed on Feb. 2, 2001.

(51) Int. Cl. G01N 33/53; C07H 21/04; C12P 21/04; C12N 5/06; C07K 14/435

(52) U.S. Cl. 435/69.7; 435/320.1; 435/325; 435/7.5; 530/350; 536/23.5

(57) ABSTRACT

The invention relates to compositions and methods for expressing a composite receptor on the cell surface. The composite receptor can be integrated into a cell membrane via a fusion peptide which includes a cell penetrating domain linked to a transmembrane domain. In a preferred embodiment, the composite receptor further comprises a ligand binding domain. In yet another embodiment the invention relates to compositions and methods for translocating a nucleic acid or other molecule across the cell membrane into the cell. In a preferred embodiment, the nucleic acid or other molecule is linked to a fusion peptide comprising an adapter domain which is linked to a cell penetrating domain.

### CROSS REFERENCE TO RELATED APPLICATIONS

The present application claims priority under 35 U.S.C. § 119(e) to U.S. Provisional Application No. 60/265,624.

### STATEMENT REGARDING FEDERALLY SPONSORED RESEARCH AND DEVELOPMENT

This invention was supported in part using funds obtained from the U.S. Government (department of Energy, Grant No. DE/FG02/00ER/63058). The U.S. Government may therefore have certain rights in this invention.

### BACKGROUND OF THE INVENTION

In many cells, the absence of appropriate membrane receptors is a major barrier to efficient ligand binding and is the primary limitation in implementing a number of potentially powerful receptor-mediated techniques as universal tools for studying events in the cell such as virus-mediated gene transfer, receptor-mediated modulation of cellular death, antibody-antigen recognition, etc. The routine approach is to transfect the cell with a gene encoding a particular receptor. This approach is time consuming and often unsuccessful because many cells, especially non-dividing cells, are poorly transfectable.

Recently, synthetic monosaccharides have been used to create a unique ketone group on the cell surface which was then used as a functional group to which other molecules were attached (Lee et al., 1999, J. Biol. Chem. 274:21878-21884). To obtain expression of the unique ketone group on the cell surface, synthetic monosaccharides were fed to cells in vitro resulting in expression of the unique ketone group on the cell surface. A foundation on which a receptor could be built was established by covalently binding biotin hydrazide to the ketone. While this method can be advantageous in some instances, it can adversely effect cell metabolism, is time consuming and has obvious limitations for in vivo use.

On the other hand, the technique known as “protein painting” exploits glycosylphosphatidylinositol (GPI)-anchor proteins, which when added to cells in vitro insert their GPI moiety directly into cell membranes to form receptors. This approach is not dependent on cellular metabolic activity (Tykocindki et al., 1996, Am. J. Pathol. 148:1-16). Obtaining GPI-anchored proteins, however, is complicated, including isolation and purification of these proteins. It also should be noted that compartmentalization of GPI-anchored proteins in the cell membrane could be considered a limiting factor for their use (Hoessli et al., 1998, Trends Cell Biol. 8:87-89).

Most biopolymers do not readily translocate across biological membranes. However, some transactivating factors and homeoproteins have been shown to be capable of facilitating membrane translocation, including Tat derived peptides (Fawell et al., 1994 Proc. Natl. Acad. Sci. USA 91:664-668), the third helix of the antennapedia homeodomain protein (Derossi et al., 1994, J. Biol. Chem. 269:10444-10450; U.S. Pat. Nos. 5,888,762 and 6,015,787), and VP22 (Schwarze et al., 2000, Trends Pharmacol. Sci. 21:45-48).

It has been demonstrated that short synthetic peptides corresponding to some specific positively charged domains of these proteins are capable of mediating translocation across the cell membrane in a membrane receptor-independent manner. Moreover, these peptides, termed “cell penetrating peptides” or “trojan peptides”, have been shown to be capable of translocating hydrophilic macromolecules across the cell membrane both in vitro and in vivo. To date, no prior studies have reported the use of cell penetrating peptides in connection with any transmembrane motifs to display biochemical moieties or receptors on the cell surface.

While translocation of peptides and proteins across cell membranes with trojan peptides is well documented (Schwarze et al., 1999 Science 285:1569-1572; Schwarze et al., 2000 Trends Pharmacol. Sci. 21:45-48; Derossi et al., 1998, Trends Cell Biol. 8:84-87; Lindgren et al., 2000, Trends Pharmacol. Sci. 21:99-103), the translocation of nucleic acids across cell membranes has not been as extensively studied. Furthermore, reports on translocation of nucleic acids or their analogs such as peptide-nucleic acid conjugates (Lindgren et al., 2000, Trends Pharmacol. Sci. 21:99-103) have involved covalent coupling of the cell penetrating peptides to the nucleic acid species. Thus, a need in the art exists for peptides with adapter domains linked to a cell penetrating peptide which permit coupling of nucleic acids to the adapter domain by electrostatic or other types of interactions which are not covalent. Such a peptide would markedly simplify the construction of translocation complexes and greatly enhance the flexibility and diversity of methods for translocation of a nucleic acid into a cell.

Many diseases, including cancer and other proliferative disorders, as well as diseases involving receptors (e.g., cystic fibrosis), can benefit from techniques which can add receptors to the cell surface. Likewise, the ability to easily transport nucleic acids into cells can benefit many diseases.

There is a long felt need in the art for the development of new methods for inserting peptides into membranes to display biochemical moieties or receptors and for the development of peptides with adapter domains to transport nucleic acids or other molecules into a cell. The present invention satisfies these needs.

### SUMMARY OF THE INVENTION

The invention relates to a fusion peptide comprising at least one cell penetrating domain linked to at least one transmembrane domain, wherein the orientation of the cell penetrating domain is independent of the orientation of the transmembrane domain. The invention also relates to a nucleic acid encoding the fusion peptide. In one aspect the cell is a eukaryotic cell. In another aspect the cell is a mammalian cell. In yet another aspect the cell is a human cell.

In one embodiment the cell penetrating domain sequences are selected from SEQ ID NOs: 1-20 and 21. In another embodiment, the invention includes a nucleic acid encoding a peptide comprising the cell penetrating domain sequences selected from SEQ ID NOs: 1-20 and 21.

In another aspect of the invention, the transmembrane domain sequences for the fusion peptide are selected from the group consisting of SEQ ID NOs: 22-27 and 45. The invention also relates to a nucleic acid encoding such sequences.

In one embodiment, the fusion peptide of the invention further comprises a ligand binding domain. The ligand binding domain may comprise a receptor. The invention may include a receptor for a peptide, a growth factor, a cytokine or a hormone. In one aspect of the invention the receptor is a receptor for peptides or proteins and in another aspect it may be a receptor for other types of molecules such as steroids. In another embodiment the ligand binding domain is an effector domain.

The invention also relates to compositions and methods for expressing a fusion peptide comprising a composite receptor on the cell surface. The invention further relates to methods of administering a fusion peptide to a cell. The method of the invention includes administering a fusion peptide comprising a composite receptor to a eukaryotic cell. The invention also relates to a nucleic acid encoding the fusion peptide. In one aspect, the composite receptors of the invention are CR1, CR1R, CR2, and delta CR1.

The invention additionally relates to a method of identifying a binding partner of a receptor domain of a composite receptor fusion peptide and to isolated binding partners of receptor domains. The method comprises contacting a cell comprising the receptor domain with a test compound, comparing the level of binding of the test compound with the cell with the level of binding of the test compound in an otherwise identical cell not comprising the receptor domain. A higher level of binding of the test compound in the cell contacted with the test compound, compared with the level of binding of the test compound in the otherwise identical cell not comprising the receptor domain is an indication that the test compound is a binding partner of the receptor domain. In one aspect, the invention includes a compound identified by the method.

The invention also relates to compositions comprising a fusion peptide or isolated peptide and a pharmaceutically acceptable carrier. In another aspect, the invention relates to a kit for administering a composite receptor peptide to a cell. In yet another aspect, the kit includes a composite receptor selected from the group consisting of Composite Receptor 1, Composite Receptor 1R, and Composite Receptor 2.

In addition, the invention relates to a fusion peptide comprising a cell penetrating domain which is linked to an adapter domain capable of binding to a molecule, wherein the orientation of the cell penetrating domain is independent of the orientation of the adapter domain. The invention further relates to an isolated nucleic acid encoding such a fusion peptide. In one aspect the cell is a eukaryotic cell. In another aspect the cell is a mammalian cell. In yet another aspect the cell is a human cell.

In another aspect, the invention relates to a fusion peptide comprising a cell penetrating domain and an adapter domain, wherein the fusion peptide is capable of facilitating translocation of a molecule across a cell membrane. The invention also includes such a fusion peptide wherein the cell penetrating domain is selected from the group consisting of SEQ ID NOs: 1-20 and 21 and the adapter domain is selected from the group consisting of SEQ ID NOs: 28-35, 47, 55, and 56. In another aspect, the invention includes an isolated nucleic acid encoding such a fusion peptide.

In another aspect of the invention, the adapter domain is a chemical moiety selected from the group of consisting of (2-dimethylamino)ethyl methacrylate, polyallylamine, hydroxyapatite, polyethyleneimine, protamine, glucaramide polymers, polyamines, and N-substituted glycine (NSG) peptoids.

In one aspect of the invention, the nucleic acid which is transported is selected from the group consisting of oligonucleotides, DNA, and RNA. In yet another aspect of the invention, the nucleic acid may be single stranded, double stranded, or a triplex, or other structure. The invention also includes a ribozyme.

The invention also relates to a fusion peptide which facilitates translocation of a nucleic acid, wherein the fusion peptide is selected from the group consisting of SEQ ID NOs: 36, 37, 41, 42, 47, 48, 49, 50 and 51.

In yet another aspect, the invention is an isolated peptide comprising an amino acid sequence selected from the group consisting of SEQ ID NOs: 41, 49, and 50. In a further aspect, the invention is an isolated peptide comprising conservative substitutions of the amino acid sequences selected from the group consisting of SEQ ID NOs: 41, 49, and 50. And in yet another aspect, the invention relates to an isolated peptide comprising a fragment of at least three amino acids of an amino acid sequence selected from the group consisting of SEQ ID NOs: 41, 49, and 50. The invention also includes naturally occurring amino acid sequence variants of amino acid sequences selected from the group consisting of SEQ ID NOs: 41, 49, and 50.

The invention also relates to a composition comprising a peptide capable of facilitating molecule transport and a pharmaceutically acceptable carrier.

In one aspect, the invention relates to a method of facilitating translocation of a molecule into a cell, wherein the method comprises administering to a cell the molecule and an effective amount of a transport facilitating fusion peptide of the invention, further wherein the fusion peptide facilitates translocation of the molecule into the cell. In another aspect of the invention the molecule is a nucleic acid. In yet another aspect of the invention the molecule transported by the fusion peptide is yet another protein or peptide or other molecule. In one aspect, the cell is a eukaryotic cell. In yet another aspect the cell is a mammalian cell. In a further aspect, the cell is a human cell.

The invention also relates to a method of identifying a fusion peptide comprising an adapter domain capable of translocating a molecule into a cell. In one aspect, the method comprises contacting a cell with a fusion peptide comprising a test adapter domain and a molecule, and then comparing the level of translocation of the molecule into the cell with the level of translocation into an otherwise identical cell contacted with the molecule. A higher level of translocation of the molecule into the cell is an indication that the fusion peptide comprising a test adapter domain is capable of translocating a molecule into a cell. In one aspect, the molecule is a nucleic acid. In yet another aspect, the nucleic acid is selected from the group consisting of the nucleic acid sequence of SEQ ID NOs: 40, 52, 53, and 54. In another aspect, the nucleic acid is an oligonucleotide comprising SEQ ID NO: 40. In an additional aspect, the invention relates to an adapter domain identified by the method of the invention. In a further aspect, the cell is a eukaryotic cell. The invention also relates to a mammalian cell. And in yet another aspect the invention includes a human cell.

The invention also relates to a kit comprising a fusion peptide of the invention capable of facilitating transport of a molecule across a cell membrane. In one aspect, the molecule is a nucleic acid.

### BRIEF DESCRIPTION OF THE DRAWINGS

FIG. 1, comprising FIG. 1A, FIG. 1B, FIG. 1C, FIG. 1D, FIG. 1E, and FIG. 1F, is a series of images of photomicrographs depicting that the composite receptors (CR), CR1 and CR2, are localized at the cell surface, while Tat-K₁₆ peptide localizes in the cytoplasm and the nucleus. The images on the left represent phase contrast photomicrographs and those on the right represent fluorescent photomicrographs. SK-BR-3 cells were incubated with fluorescein labeled Tat-K₁₆ (FIG. 1A and FIG. 1B), CR1 (FIG. 1C and FIG. 1D), or CR2 (FIG. 1 E and FIG. 1 F), DNA was counterstained with DAPI, and then the cells were subjected to fluorescence microscopy. The peptides (CR1, Tat-K₁₆ and CR2) are labeled with fluorescein and appear green/dark, while the nuclei are stained with DAPI and appear blue/dark. The borders of the cytoplasm are defined in the phase contrast images on the left. The composite receptor molecules CR1 and CR2, which contain both the cell penetrating and transmembrane domains, are localized at the periphery of SK-BR-3 cells, while the Tat-K₁₆ peptide is located throughout the cell.

FIG. 2, comprising FIG. 2A, FIG. 2B, FIG. 2C, FIG. 2D, and FIG. 2E, illustrates schematically various composite receptor structure variations. In its most basic form, the composite receptor comprises a transmembrane domain and a cell penetrating domain (FIG. 2A). In another form, the composite receptor further comprises a ligand binding domain in the extracellular domain of the composite receptor (FIG. 2B). In alternative forms the ligand binding domain can be in the intracellular domain between the transmembrane domain and the cell penetrating domain (FIG. 2C). Alternatively, the ligand binding domain can be on the end of the fusion molecule adjacent to the cell penetrating domain (FIG. 2D). The composite receptor can also comprise more than one ligand binding domain in various configurations as shown (FIG. 2E). In the Figure, the abbreviations TM, CPD, and LBD, are Transmembrane Domain, Cell Penetrating Domain, and Ligand Binding Domain, respectively.

FIG. 3, comprising FIG. 3A, FIG. 3B, and FIG. 3C, is a series of images of fluorescent micrographs illustrating the effects of a cell penetrating domain on the efficiency of composite receptors binding. SK-BR-3 cells were incubated with the fusion peptides CR1 (biotin-G₃YGRKKRRQR₃G₃AAL₃A₁₉WK₈-FITC) or delta-CR1 (FITC-G₃G₃AAL₃A₁₉WK₈-biotin) which have the same amino acid structure, but the latter has a deletion of the cell penetrating domain. FIG. 3A depicts cells which received no peptides, thus, no fluorescence appears. FIG. 3B depicts cells incubated with 200 nM delta-CR1 and it can be seen that there is a low level of fluorescence on the cell membrane. FIG. 3C depicts cells incubated with 200 nM CR1 and it can be seen that high levels of fluorescence occur on the cell membrane.

FIG. 4, comprising FIG. 4A and FIG. 4B, is a series of images of fluorescent micrographs of SK-BR-3 cells incubated with the composite receptors CR1R (biotin-G₃YGRKKRRQR₃G₃AAL₃A₁₉WK₈-FITC) (FIG. 4A) or CR1 (FITC-G₃YGRKKRRQR₃G₃AAL₃A₁₉WK₈-biotin) (FIG. 4B), which have the same amino acid structure but a different orientation of their lateral FITC groups. This difference in membrane orientation can be detected using an anti-FITC antibody which cannot penetrate the cellular membrane and therefore can only bind to an FITC on the outer cell surface. For CR1, FITC is an intracellular domain. However, for CR1R, FITC is an extracellular domain. Alexa-Fluor 594 Rabbit polyclonal anti-FITC antibody (Molecular Probes) was used to detect receptor orientation in the cellular membrane. SK-BR-3 cells were collected and washed in DPBS with 0.5% BSA (DPBS-BSA), resuspended in DPBS-BSA with 10 mM Sodium Azide, incubated for 20 minutes at room temperature, transferred to ice. 5×10⁴ cells were mixed with 60 nM CR1 or CR1R in 20 μl DPBS-BSA, incubated 30 min on ice, washed in DPBS-BSA -Azide, and then resuspended in 50 μl DPBS-BSA-Azide. Then, 50 μl of anti-antibody in DPBS-BSA-Azide (dilution 1:50) was added, the suspension incubated on ice for 30 minutes, and then the cells were washed and applied to slides. Alexa-Fluor 594 fluorescence was analyzed by microscope using monochrome filter for Cy3.5. It can be seen that the fluorescence of anti-fluorescein antibodies is higher for CR1R peptide which has the extracellular orientation of the FITC group (FIG. 4B).

FIG. 5, comprising FIG. 5A and FIG. 5B, is a series of images of photomicrographs illustrating the fact that both Tat (FIG. 5A) and Tat-K₁₆ (FIG. 5B) peptides enter cells and that upon entry into a cell each is located in both the nucleus and cytoplasm. SK-BR-3 human adenocarcinoma cells were incubated with fluorescein labeled Tat or Tat-K₁₆ for 15 minutes, the DNA was counterstained with DAPI, and images were obtained using an epifluorescence microscope. The DNA counterstain is shown in blue and the peptide-FITC in green.

FIG. 6 is a series of images of photomicrographs illustrating the translocation of an oligonucleotide with Tat-K₁₆, but not Tat. Tat-K₁₆ is a fusion peptide which comprises an adapter domain, while Tat is a peptide which does not comprise an adapter domain. Tat or Tat-K₁₆ peptides were mixed with a fluorescent labeled Cy3-oligonucleotide (5′-Cy3-TATATGATGGRTACCGCAG-‘3dT-5’; SEQ ID NO: 40). SK-BR-3 human adenocarcinoma cells were incubated with the mixture and then subjected to fluorescence microscopy. The relative distributions of the fluorescein isothiocyanate (FITC)-Tat-K₁₆ or FITC-Tat peptides are shown in green, and the Cy3-oligonucleotide (5′-Cy3-TATATGATGGRTACCGCAG-‘3dT-5’; SEQ ID NO: 40) is shown in red. The left column depicts merged images from a cell treated with both Tat-K₁₆ and Cy3 oligonucleotide, the middle column depicts merged images from a cell treated with both Tat and Cy3 oligonucleotide, and the right column depicts merged images from a cell treated only with Cy3 oligonucleotide. The upper panel represents Tat signal, the middle panel represents the oligo signal, and the lower panel represents the DAPI signal. It can be seen that the only group in which the oligonucleotide is present in the cells is the group incubated in the presence of both Tat-K₁₆ and the oligonucleotide (left column, middle row).

FIG. 7 is a graph depicting the peptide mediated uptake of the fluorescent-labeled oligonucleotide FITC-O-24 at various peptide concentrations. HeLa cells were incubated with a mixture of the 24-mer oligonucleotide FITC-O-24 and a peptide (K16, TAT, TAK8, TAK12, or TAK16). The ordinate represents units of fluorescence and the abscissa represents the concentration (micromolar) of the particular peptide used.

FIG. 8 is a series of images of photomicrographs depicting TAK-16 peptide mediated uptake of the 24-mer oligonucleotide (FITC-O-24) into HeLa cells as determined by fluorescence microscopy. TAK-16 was used at 0.00 μM, 0.36 μM, 1.80 μM, or 9.00 μM. FITC-O-24 was present at 2.5 μM in each sample. The cells in the upper panels were stained with DAPI (blue fluorescence) to reveal DNA while the cells of lower panels were measured for FITC-oligonucleotide uptake (green fluorescence).

FIG. 9 is a graph illustrating TAK-16 peptide mediated uptake of an 83-mer oligonucleotide, as measured by flow cytometry. HeLa cells were incubated with 13.3 μM TAK-16 peptide and various concentrations of the FITC-labeled 83-mer and then subjected to flow cytometry. The ordinate represents cell number. The abscissa represents the intensity of fluorescence and above each peak is indicated the concentration of FITC-labeled oligo (O-83-FITC) associated with the corresponding peak of fluorescent intensity, namely 0.00 μM, 0.02 μM, 0.07 μM, 0.2 μM, 0.6 μM, and 1.8 μM.

FIG. 10, comprising FIGS. 10A, 10B, 10C, 10D, 10E, and 10F, is a series of images of fluorescent micrographs depicting the effects of TAK-16 peptide on the uptake of an 83-mer oligonucleotide. The HeLa cell samples used in FIG. 9 were also subjected to fluorescence microscopic analyses. The concentrations of oligonucleotides used comprised 0.00 μM, 0.02 μM, 0.07 μM, 0.2 μM, 0.6 μM, and 1.8 μM (FIGS. 10A-10F, respectively).

FIG. 11, comprising FIGS. 11A, 11B, and 11C, is a series of images of photomicrographs illustrating TAK-16 peptide mediated uptake of Cy3 labeled linear double stranded (DS) 400 base pair (bp) DNA and circular 5kb plasmid DNA (pECFP). HeLa cells were incubated with Cy3 labeled 400 bp DNA (60 ng) or plasmid DNA (400 ng) and TAK-16 at 12 μM. The cells were then subjected to fluorescence microscopic analyses. FIG. 11A depicts control cells, e.g., cells which were incubated with plasmid DNA and no peptide; FIG. 11B depicts cells incubated with 400 bp DNA and TAK-16; and FIG. 11C depicts cells incubated with plasmid DNA and TAK-16.

FIG. 12, comprising FIGS. 12A and 12B, is a series of images of fluorescent photomicrographs demonstrating TAK-16 mediated uptake of biotinylated oligo-avidin-FITC complex. FIG. 12A is an image of a fluorescent micrograph of control HeLa cells which were incubated with TAK16 and avidin-FITC, but without the oligonucleotide. FIG. 12B is an image of a fluorescent micrograph of HeLa cells incubated with TAK-16, a 22-mer biotin labeled oligonucleotide (Bio-O-22), and an avidin-FITC conjugate, which demonstrates that TAK-16 in the presence of the oligonucleotide mediates the uptake of the avidin conjugate.

FIG. 13, comprising FIGS. 13A and 13B, is two images of photomicrographs depicting cytochemical analyses of TAK-16-oligo vector mediated uptake of horseradish peroxidase. HeLa cells were incubated with horseradish peroxidase-avidin (HPA), Bio-O-22 oligonucleotide, and/or TAK-16 peptide. Cytochemical analysis reveals that TAK-16 mediated uptake of HPA (brown/dark staining) in the cells incubated with all three components (FIG. 13B), while there was no uptake in cells incubated without the oligonucleotide (FIG. 13A).

### DETAILED DESCRIPTION OF THE INVENTION

General Description

The invention relates generally to compositions and methods for incorporating a composite receptor into a cell membrane both in vivo and in vitro. The invention relates specifically to compositions of fusion peptides comprising a cell penetrating domain linked to a transmembrane domain which is fused to a ligand binding domain. The methods of the invention include use of these fusion peptides to incorporate ligand binding sites into cell membranes.

The invention also relates to compositions and methods for translocating a nucleic acid or other molecule across a cell membrane into the cytoplasm and further including transport of the molecule into the nucleus as well. The invention relates specifically to compositions of fusion peptides comprising a cell penetrating domain linked to an adapter domain capable of binding nucleic acids. In the present invention it has been discovered that a cell penetrating fusion peptide which has an adapter domain capable of binding a nucleic acid can transport a nucleic acid into a cell. Methods of the present invention which use this latter composition include use of the fusion peptides to transduce a cell with a nucleic acid. It has also been discovered in the present invention that a fusion peptide and a nucleic acid can be used to facilitate transport of yet another protein or other molecules across the cell membrane.

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

As used herein, “alleviating a disease or disorder symptom” means reducing the severity of the symptom.

As used herein, “amino acids” are represented by the full name thereof, by the three-letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Praline | Pro | P |
| Phenylalanine | Phe | F |
| Tryptophan | Trp | W |

As used herein, the term “adapter domain” refers to the region of a fusion peptide which links a cell penetrating domain to a nucleic acid or other molecule. These domains include, but are not limited to, the amino acid sequences and chemical moieties presented in Table 3.

The term “binding” refers to the adherence of molecules to one another, such as, but not limited to, enzymes to substrates, ligands to receptors, antibodies to antigens, DNA binding domains to DNA, and DNA or RNA strands to complementary strands.

“Binding partner,” as used herein, refers to a molecule capable of binding to another molecule.

As used herein, the term “cell penetrating domain” refers to a peptide capable of directly permeating the cell membrane in a receptor- or transporter-independent manner and which is used to transport attached molecules through the cell membrane into a cell. These domains include, but are not limited to, the peptide sequences disclosed in Table 1. This term is used synonymously with “membrane translocation domain” and “trojan peptide” and “penetratins” and the like. Attached molecules include, but are not limited to, peptides, proteins, nucleic acids, polysaccharides, ligands, cofactors, chemical moieties and the like.

As used herein, the term “composite receptor” refers to a membrane or cell-surface receptor or ligand binding domain which comprises at least one cell penetrating domain and at least one transmembrane domain.

A “compound,” as used herein, refers to any type of substance or agent that is commonly considered a drug, or a candidate for use as a drug, as well as combinations and mixtures of the above.

As used herein, the terms “conservative variation” or “conservative substitution” refer to the replacement of an amino acid residue by another, biologically similar residue. Conservative variations or substitutions are not likely to significantly change the shape of the peptide chain. Examples of conservative variations, or substitutions, include the replacement of one hydrophobic residue such as isoleucine, valine, leucine or alanine for another, or the substitution of one charged amino acid for another, such as the substitution of arginine for lysine, glutamic for aspartic acid, or glutamine for asparagine, and the like.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, the term “effector domain” refers to a domain capable of directly interacting with a effector molecule, chemical or structure in the cytoplasm which is capable of regulating a biochemical pathway.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “extracellular domain” refers to a region of a molecule or structure that is outside the cell, the remainder of the molecule or structure being in the cell membrane and inside the cell.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment”, as applied to a nucleic acid, can ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, the term “fusion peptide” refers to a heterologous peptide, polypeptide or protein which contains a cell penetrating domain linked to one or more transmembrane domains, ligand binding domains and/or adapter domains. Each domain can comprise amino acid residues or chemical moieties which mimic the structure of such residues also known as peptide mimetics. Each domain can be linked to another domain covalently or non-covalently. The covalent linkage can be stable or labile to allow disconnection of a domain from the peptide if desired. The term “fusion” as used herein is synonymous with “chimeric” or “hybrid” as used throughout the specification. The peptide can be synthetic or naturally occurring.

The term “growth factor,” as used herein, refers to those factors commonly known in the art which are capable of eliciting growth or other responses from cells.

“Hormone,” as used herein, has the common meaning of a factor which generally acts at a site distant to where it is produced. However, the term also now encompasses known hormones which may act locally.

As used herein, the term “heterologous peptide” refers to any peptide, polypeptide or protein whose sequence is chosen in such a way that the product of the fusion of this sequence with one or more transmembrane domains, ligand binding domains, adapter domains and/or cell penetrating domains results in a sequence different from the wild-type sequence flanking any of these domains.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound invention or be shipped together with a container which contains the identified compound. Alternatively, the instructional material can be shipped separately from the container with the intention that the instructional material and the compound be used cooperatively by the recipient.

As used herein, the term “intracellular domain” refers to a part of a molecule or structure that is inside the cell, the remainder of the molecule or structure being in the cell membrane and/or outside the cell.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g, as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats can be used to select antibodies specifically immunoreactive with a particular protein. For example, solid-phase ELISA immunoassays are routinely used to select monoclonal antibodies specifically immunoreactive with a protein. See Harlow and Lane 1988, Antibodies, A Laboratory Manual, Cold Spring Harbor Publications, N.Y.) for a description of immunoassay formats and conditions that can be used to determine specific immunoreactivity.

As used herein, the term “ligand binding domain” refers to the reactive part of a macromolecule that directly participates in its specific combination with another molecule. This term includes local surface sites on peptides, polypeptides and proteins such as ligand binding domains which interact with contact sites on other molecules, including small molecules, macromolecules, nucleic acids, peptides, polypeptides or proteins. A ligand binding domain can also serve as an “effector domain”, as set forth herein, when it is intracellularly located.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “peptide” typically refers to short polypeptides.

As used herein, the term “pharmaceutically-acceptable carrier” means a chemical composition with which an appropriate fusion peptide or derivative can be combined and which, following the combination, can be used to administer the appropriate fusion peptide to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

A “polynucleotide” means a single strand or parallel and anti-parallel strands of a nucleic acid. Thus, a polynucleotide may be either a single-stranded or a double-stranded nucleic acid.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but can be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or exhibits only early signs of the disease for the purpose of decreasing the risk of developing pathology associated with the disease.

The term “protein” typically refers to large polypeptides.

“Synthetic peptides or polypeptides” mean a non-naturally occurring peptide or polypeptide. Synthetic peptides or polypeptides can be synthesized, for example, using an automated polypeptide synthesizer. Those of skill in the art know of various solid phase peptide synthesis methods.

A “therapeutic” treatment is a treatment administered to a subject who exhibits signs of pathology, for the purpose of diminishing or eliminating those signs.

A “therapeutically effective amount” of a compound is that amount of compound which is sufficient to provide a beneficial effect to the subject to which the compound is administered.

As used herein, the term “transmembrane domain” refers to the domain of a peptide, polypeptide or protein which is capable of spanning the plasma membrane of a cell. These domains can be used to anchor a composite receptor on the cell membrane surface. The term is used synonymously with “membrane anchor domain” and the like.

The term to “treat,” as used herein, means reducing the frequency with which symptoms are experienced by a patient or subject or administering an agent or compound to reduce the frequency with which symptoms are experienced.

As used herein, “treating a disease or disorder” means reducing the frequency with which a symptom of the disease or disorder is experienced by a patient. Disease and disorder are used interchangeably herein.

As used herein, the term “wild-type” refers to the genotype and phenotype that is characteristic of most of the members of a species occurring naturally and contrasting with the genotype and phenotype of a mutant.

Fusion Peptides with Transmembrane Domains

The present invention relates to compositions and methods for inserting a peptide of interest into the plasma membrane of cells. In one embodiment, the composition is a fusion peptide which is a composite receptor comprising at least one transmembrane domain linked to a cell penetrating domain. The specific order of these domains can be established from the amino and carboxy termini with the transmembrane domain starting at the amino terminus and the cell penetrating domain ending at the carboxy terminus, as well as in the opposite orientation.

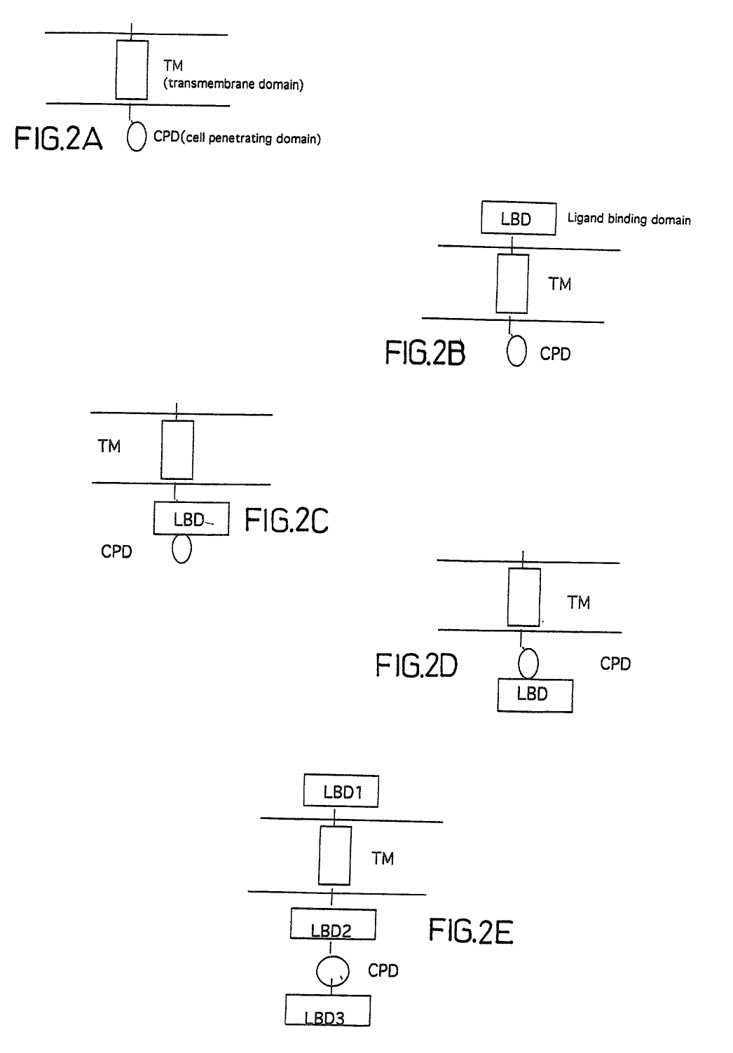

In its most basic form, the composite receptor comprises a transmembrane domain and a cell penetrating domain (FIG. 2). In another form, the composite receptor further comprises a ligand binding domain in the extracellular domain of the composite receptor. In alternative forms the ligand binding domain can be in the intracellular domain between the transmembrane domain and the cell penetrating domain. Alternatively, the ligand binding domain can be on the end of the fusion molecule adjacent to the cell penetrating domain. The composite receptor can also comprise more than one ligand binding domain in various configurations (FIG. 2).

In one embodiment, the composite receptor further comprises at least one extracellular domain. The specific order of these domains can be established from the amino and carboxy termini with the cell penetrating domain starting at the amino terminus and the transmembrane domain ending at the carboxy terminus or vice versa. The composite receptor can comprise at least one ligand binding domain which can be in either the intracellular or extracellular domain of the composite receptors. Composite receptors are most commonly used to “paint” the cell surface with a particular receptor in a rapid manner, preferably in several minutes, using the methods of the invention.

Each domain can be linked to its adjacent domains either covalently or non-covalently. The covalent linkage can be stable or labile to allow separation of a domain following insertion if desired.

The intracellular domain comprises at least one cell penetrating domain which crosses the cell membrane inserting the transmembrane domain into the plasma cell membrane such that it is integrated into the lipid bilayer. The transmembrane domain does not enter into the cytoplasm but remains in the membrane and anchors the fusion peptide in the membrane such that the intracellular domain is exposed on the intracellular surface of the membrane. A common feature of all cell penetrating domains is that they have abundant numbers of positively charged amino acids. In a preferred embodiment, the intracellular domain comprises a cell penetrating domain which is derived from any one of the sequences set forth in Table 1. The invention should not be construed to include only the cell penetrating domain sequences of Table 1, but should be construed to include other cell penetrating domain sequences as well.

In another embodiment, the intracellular domain further comprises an effector domain capable of interaction in signal transduction in the cytoplasm of the cell. Such effector domains are capable of interacting with cytoplasmic macromolecules thereby effecting signal transduction events such as receptor-effector coupling following ligand binding to a ligand binding domain in the extracellular domain of a composite receptor.

TABLE 1

Cell penetrating domain sequences

| Protein (residues) | Amino Acid Sequence | SEQ ID | Reference |
|---|---|---|---|
| HSV-1 VP22 | DAATATRGRSAASRPTERPRAPARSA | 1 | Schwarze and Doody, |
| | SAPAAPVG | | 2000, Trends |
| ANTP | RQIKIWFQNRRMKWKK | 2 | Pharmacol. Sci. 2L45- |
| | | | 48 |
| D-Tat | GRKKRRQRRRPPQ | 3 | Futaki et al., 2001, J. |
| HIV-1 Tat (48-60) | GRIKKIRRQRRRPPQ | 4 | Biol. Chem. |
| | | | 276:8:5836-5840 |
| R9-Tat | GRRRRRRRRRPPPQ | 5 | |
| U2AF (142-153) | SQMTRQARRLYV | 6 | |
| HIV-1 Rev (34-50) | TRQARRNRRRRWRERRQR | 7 | |
| FHV Gacoat | RRRRNRTRRNRRRVR | 8 | |
| (35-49) | | | |
| BMV Gag (7-25) | KMTRAQRRAAARKNKRNTR | 9 | |
| HTLV-II Rex | TRRQRTRRARRNR | 10 | |
| (4-16) | | | |
| CCMV Gag (7-25) | KLTRAQRRAAARKNKRNTR | 11 | |
| P22 N (14-30) | NAKTRRHERRRKLAIER | 12 | |
| cFos (139-164) | KRRIRRERNKMAAAKSRNRRRELTD | 13 | |
| | DT | | |
| cJun (252-279) | RIKAERKRMRNRIAASKSRKRKLERI | 14 | |
| | AR | | |
| GCN4 (231-252) | KRARNTEAARRSRARKLQRMQK | 15 | |
| PTD-4 | PIRRRKIKLRRLK | 16 | Mi et al., 2000, Mol. |
| PTD-5 | RRQRRTSKLMKR | 17 | Ther. 2:339-347 |
| Penetratin | GRKKRRQRRRPPQ | 18 | Lindgren et al., 2000 |
| Transportan | GWTLNSAGYLLKINLKALAALAALIL | 19 | Trends Pharmacol. |
| Amphipathic | KLALKLALKALKAALKLA | 20 | Sci. 21:99-103 |
| peptide | | | |
| HIV-1 Tat (47-58) | YGRKKIRRQRRR | 21 | Fawell et al., 1994, |
| | | | Proc. Natl. Acad. Sci. |
| | | | USA 91:664-668 |

In the case of composite receptors further comprising an extracellular domain, the extracellular domain can comprise a ligand binding domain. The ligand binding domain is generally a protein or peptide, a nucleotide sequence, or other chemical which has diagnostic, prophylactic or therapeutic application (referred to herein as a drug). The ligand binding domain is linked, as described below, with a transmembrane domain which in turn is linked to a cell penetrating domain.

In a preferred embodiment, the extracellular domain comprises a ligand binding domain which is capable of interacting with contact sites on other molecules, including but not limited to small molecules, macromolecules, nucleic acids, peptides, polypeptides or proteins. In another embodiment, interaction of a molecule with the extracellular domain of the invention can elicit an intracellular response.

In one embodiment, the composite receptor comprises a ligand binding domain where the ligand is any molecule which is presented to the extracellular surface.

In one embodiment of the present invention, the ligand binding domain is a receptor protein whose expression on the plasma cell membrane is desired. Receptors are molecules which selectively interact with other molecules. Receptor molecules can perform a variety of tasks from selective binding of substrates and transduction of a signal across the cell membrane to catalyzing chemical reactions. One example of a receptor molecule is an antibody. Monoclonal antibodies bind to other molecules (antigens) with very high selectivity, while in other instances they catalyze chemical reactions by selectively binding the transition states of those chemical reactions. Monoclonal antibodies are used as medicinal and diagnostic agents.

Cell surface receptors include, but are not limited to, receptors for growth factors, cytokines, and hormones.

Other receptor molecules are used as drug targeting molecules and are sometimes referred to as “magic bullets” which can be used to target a particular cell. In all cases, the effectiveness of a receptor molecule is dependent upon its ability to bind molecular species (substrates) with high discrimination and selectivity (i.e., not bind other, often closely related, molecular species).

In a further embodiment, the extracellular domain is a drug, immunogenic peptide, epitope or enzyme binding motif, such as a peptide analog or small molecule enzyme inhibitor, whose expression on the cell membrane is desired. In yet another embodiment, the extracellular domain is a chemical or biochemical moiety to be used as a diagnostic tool. For example, the chemical moiety can be biotin which could be used to detect or target cells where the fusion peptide has been inserted into the cell membrane by taking advantage of the high binding affinity this molecule displays for avidin or streptavidin.

In another embodiment, the extracellular domain is a nucleic acid sequence to be used as a diagnostic tool or probe, or as a therapeutic agent, such as a nucleic acid sequence which can be amplified using the polymerase chain reaction or other amplification techniques (e.g., rolling circle amplification (RCA), tyramide signal amplification (TSA) and the like) to detect cells which are expressing the ligand binding domain.

Alternatively, the polymerase chain reaction can be used to detect or identify the presence of cells expressing the ligand binding domain in biological fluids following in vivo administration of a fusion peptide comprising an extracellular ligand binding domain, which further comprises a nucleic acid. In yet another embodiment, complementary nucleic acid probes which hybridize to the nucleic acid receptors can be used to detect cells expressing the ligand binding domains. The polymerase chain reaction should not be construed to be limited solely to the assays described herein to detect or identify the presence of cells expressing a ligand binding domain. The invention should also be construed to include other assays as well.

Any ligand binding domain can be delivered to the cell membrane using the compositions and methods of the subject invention. For example, in one embodiment of the present method, the molecule to be delivered to the cell membrane is a protein, a peptide, an oligonucleotide, RNA or DNA. The invention should not be construed to include only these types of molecules as being capable of being delivered to the cell membrane, but should be construed to include other types of molecules as well.

The present invention is particularly useful for inserting proteins or peptides, such as regulatory factors, enzymes, antibodies, drugs or toxins, and nucleic acids such as DNA or RNA, into the cell membrane. In one embodiment, the ligand binding domain is an epitope for a particular antibody or antigenic determinant for facilitating a particular type of immune response.

The transmembrane domain of the fusion peptides of the invention can be any molecule which spans the plasma cell membrane and can anchor other domains to the membrane. A transmembrane domain may comprise hydrophobic regions or amphipathic regions. Hydrophobic regions contain hydrophobic amino acids which include, but are not limited to, phenylalanine, methionine, isoleucine, leucine, valine, cysteine, tryptophan, alanine, threonine, glycine and serine and include hydrophobic alpha-helices.

Amphipathic regions may have both hydrophobic and hydrophilic amino acids and moieties and include amphipathic alpha-helices. Hydrophilic amino acids include, but are not limited to, arginine, aspartate, lysine, glutamate, asparagine, glutamine, histidine, tyrosine and proline. Transmembrane domains which form stable alpha helices have been previously described and include peptides comprising the sequences in Table 2. The invention should not be construed to be limited solely to the transmembrane domain sequences listed in Table 2, but should be construed to include other transmembrane domain sequences as well.

TABLE 2

Transmembrane domain sequences

| | | | |
|---|---|---|---|
| Protein | Amino Acid Sequence | SEQ ID | Reference |

| | | | |
|---|---|---|---|
| CR1 | AALLLA₁₉W | 22 | Percot et al., 1999, |
| | | | Biopolymers 50:647- |
| | | | 655 |
| P24 | K₂GL₂₄K₂A | 23 | Zhang et al., 1995, |
| | | | Biophys. J. 66:847-857 |
| HIV-1 Vpu | IAIVALVVAIIIAIVVWSIVIIEY | 24 | Wray et al., 1999, |
| | | | Biochemistry 38:5272- |
| | | | 5282 |
| CXCR4-2-2 | LLFVITLPFWAVDAVANWYFGNDD | 25 | Tarasova et al., 1999, J. |
| | | | Biol. Chem. |
| | | | 274:34911-34915 |
| CR2(E5) | YVLFFLLLFLLLLLQMAAVLGLFLLF | 26 | Klein et al., 1999, J. |
| | | | Virol. 73:3264-3272 |
| EGF TMP | IATGMVALLLLLVVALGIGLFM | 27 | Jones et al., 1998 |
| | | | Biochemistry |
| | | | 37:16780-16787 |

Uses for Fusion Peptides with Transmembrane Domains

In one embodiment, the present invention provides methods for isolating and identifying binding partners of a receptor protein domain or ligand binding domain. Ligand-receptor interactions control many essential normal cellular responses, and aberrancies in these interactions and responses are implicated in many diseases and disorders. Because not all binding partners for every known ligand or receptor have been identified, there is a need to identify undiscovered binding partners. In one aspect of the embodiment, a method of identifying a binding partner of a receptor domain of a composite receptor fusion peptide, comprises contacting a cell comprising said receptor domain with a test compound, comparing the level of binding of said test compound with said cell with the level of binding of said test compound in an otherwise identical cell not comprising said receptor domain, wherein a higher level of binding of said test compound in said cell contacted with said test compound, compared with the level of binding of said test compound in said otherwise identical cell not comprising said receptor domain, is an indication that said test compound is a binding partner of said receptor domain. Assays useful in measuring such tests are described herein and if not described herein are known those of skill in the art.

In another embodiment, the method of the invention is useful in testing the result of interaction of an agent with the ligand binding site of a receptor in order to identify inhibitors or stimulators of ligand-receptor induced signal transduction. The present invention therefore includes methods of screening for agents which activate, or act as agonists, of a receptor. The present invention also includes methods of screening for agents which deactivate, or act as antagonists of a receptor. Such agents can be useful in the modulation of pathological conditions associated with modulation of receptor protein activity or expression levels. Many assays are known to those of skill in the art which can be used to test the result of ligand-receptor interaction, which assays depend on the particular ligand-receptor interaction being studied. For example, a test agent can be administered to a cell comprising a fusion peptide further comprising a receptor domain or ligand binding domain of interest, the level of stimulation or inhibition of the response of interest can be measured and compared with the level of stimulation or inhibition of the response in an otherwise identical cell not comprising the receptor or ligand binding domain of interest.

In one embodiment, the invention disclosed herein provides a method for identifying an agent which modulates a ligand-receptor signal transduction response by administering a test agent to a cell, comparing the level of said signal transduction in said cell with the level of said signal transduction in an identical cell not administered the test agent, wherein a change in the signal transduction response is an indication that the agent modulates the response. One of skill in the art would appreciate that this type of method could be used in conjunction with many known assays to measure both stimulation and inhibition of a signal transduction response.

A skilled artisan would also appreciate, based on the disclosure provided herein, that numerous assays can be used measure the changes caused by a test agent. In one aspect, the invention discloses assays for measuring the effects of regulators of signal transduction pathways both in vivo and in vitro. These assays include, but are not limited to, sampling cells, conditioned media, tissues, and blood. These assays also include, but are not limited to, methods to measure changes in phosphorylation of transduction intermediaries, changes in expression levels of components of a signal transduction pathway (e.g., western blot analyses, far-western analyses, immunocytochemistry, inimunofluorescence, kinase assays, etc.), proliferation, etc.

A compound or agent identified by any of the disclosed assays may be administered to any animal, including a human. The compound or agent may be administered via any suitable mode of administration, such as intramuscular, oral, subcutaneous, intravenous, intravaginal, rectal, intranasal, or intradermal administration. The preferred modes of administration are oral, intravenous, subcutaneous, intramuscular or intradermal administration. The most preferred mode is subcutaneous administration. The invention contemplates the use of identified agents which bind to ligand binding domains and regulate a signal transduction response in animals. Preferably the animal is human.

In another embodiment, the receptor may be a protein which mediates entry of a virus into the cell, thereby facilitating virus-mediated gene transfer. In yet another embodiment, the receptor may be a protein which is used in the area of signal transduction research to determine what effector molecules interact with the receptor. The receptor can also be an antigen which may be used to study modulation of antigen presentation. In an alternative embodiment, the receptor may be used for receptor-mediated cell targeting.

One skilled in the art would appreciate, based upon the disclosure provided herein, that additional uses of the fusion peptides with transmembrane domains include, but are not limited to, formation of heterodimers and heteromultimers on the cell surface; mediation of intracellular signal transduction through interaction of the intracellular domain with an effector protein; and modulation of receptor and effector phosphorylation on the cell surface and in the cytoplasm. In one embodiment, expressing dimers or multimers of domains on the cell surface may be useful in eliciting stronger or longer responses (e.g., immune responses, signal transduction, etc.) than may be elicited when only monomers are present or when only a low number of the molecules or the monomer are present, or it may be useful in diagnostic assays to strengthen a signal when the assay being used is a detection assay. Administration to cells of fusion peptides comprising transmembrane domains and domains capable of eliciting a signal transduction response may be useful in several ways. In one embodiment, it may increase the strength and duration of the response of a signal response in cells which are deficient in their ability to mount such a response. In another embodiment, administering such fusion peptides capable of eliciting a signal transduction response may be useful in cells which do not normally express such peptides, because it would allow manipulation or induction of a response not typically seen in that cell.

The compositions and methods of the present invention are useful to deposit a composite receptor protein on the extracellular surface of the cell membrane either in vivo or in vitro. In one embodiment, cells which have been treated with a composite receptor fusion peptide in vitro can then be introduced into an animal to expose the expressed composite receptor molecules to an in vivo environment, which in turn allows the association of potential binding partners with the receptor protein. After binding, the peptides, polypeptides, proteins or other molecules that have become associated with the composite receptor of the invention can be isolated with, or from, the carrier cells and further analyzed. The analyses would include tests to determine the function of the identified molecule. Various assays, as described above, or are further described herein, or are known in the art, may be used to elucidate the function of the identified molecule. These assays include, but are not limited to, responses measurable by cellular assays (e.g., proliferation, motility, adhesion), biochemical assays (e.g., signal transduction, protein expression and regulation), and molecular assays (e.g., transcription, PCR), etc.

In another embodiment, cells expressing a composite receptor fusion peptide are mixed in vitro with a potential binding partner or an extract or fraction of a cell under conditions which allow the association of potential binding partners with the receptor protein. After mixing, the peptides, polypeptides, proteins or other molecules that have become associated with a protein of the invention are separated from the mixture. The binding partner bound to the receptor protein of the invention can then be removed and further analyzed. The analyses can include biochemical assays known to those of skill in the art to determine the physical nature, size, and biological properties of the newly discovered binding partner or agent, as well as the assays described herein. In another embodiment, in order to identify and isolate a binding partner, the entire receptor protein can be used. Alternatively, in yet another embodiment of the invention, a fragment of the receptor protein can be used.

Many of the biochemical and molecular methods of the invention described herein require the use of cell extracts. A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract can be mixed with cells containing the composite receptor under conditions in which association of the receptor protein with the binding partner can occur. A variety of conditions can be used, the most preferred being conditions that closely resemble conditions found in the cytoplasm of a human cell. Conditions such as osmolarity, pH, temperature, and the concentration of cellular extract used, can be varied to optimize the association of the protein with the binding partner.

After mixing under appropriate conditions, the bound complex can be separated from the rest of the mixture. A variety of techniques can be utilized to separate the components of the mixture. For example, in one embodiment, antibodies specific to a protein can be used to immunoprecipitate the binding partner complex. Alternatively, standard chemical and physical separation techniques such as chromatography and density-sediment centrifugation can be used. After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the composite receptor protein can be immobilized on a solid support. For example, the protein can be attached to a nitrocellulose matrix or acrylic beads. Attachment of the protein to a solid support aids in separating peptide-binding partner pairs from other constituents found in the extract. The identified binding partners can be either a single protein or a complex made up of two or more proteins.

Alternatively, binding partners can be identified using assays including, but not limited to, a Far-Western assay according to the procedures of Takayama et al., 1997, Methods Mol. Biol. 69:171-184 or Sauder et al., 1996, J. Gen. Virol. 77:991-996 or identified through the use of epitope tagged proteins or GST fusion proteins.

Another embodiment of the present invention provides methods for identifying agents that modulate at least one activity of a receptor protein. The activity may be any of the types of activities or signal transduction responses described herein, or known to those of skill in the art. The activity should not be construed to include only an activity described herein, but should be construed to include other types of activity as well. Such methods or assays can utilize any means of monitoring or detecting the desired activity. The skilled artisan would appreciate the various methods that can be used based on that which is known in the art and the disclosure which is provided herein. The activity can be the result of modulation that inhibits or stimulates the receptor. In one embodiment, a test agent is administered to a cell comprising a composite receptor fusion peptide and the level of activity of interest is compared to that in an otherwise identical not contacted with the test agent. When a test agent modulates the activity of interest, it has now been identified as an agent which modulates at least one activity of a receptor protein. A skilled artist would recognize that more than one activity may exist that can be utilized to identify an agent which modulates activity of a receptor protein.

In one embodiment, the relative amounts of a receptor protein of a cell population which has been exposed to the test agent can be compared to an un-exposed control cell population. Such assays measuring levels or expression of receptors can be useful in determining the biological effects of a test agent on a cell expressing a receptor of the invention. In this format, probes such as specific antibodies may be used to monitor the differential expression of the protein in the different cell populations. Cell lines or populations of cells may be exposed to the test agent under appropriate conditions and time. Cellular lysates may be prepared from the exposed cell line or population of cells and a control, unexposed cell line or population of cells. The cellular lysates may then be analyzed as described herein, or using methods known to those of skill in the art. The invention should not be construed to be limited solely to the uses described above, but should be construed to include other uses as well.

Methods for Preparing Fusion Peptides Comprising a Transmembrane Domain

The fusion peptides of the invention may be obtained or produced using known techniques, such as chemical synthesis and genetic engineering methods. The cell penetrating domain can be combined with or attached to the transmembrane domain which in turn can be attached to the ligand binding domain. In one embodiment, the transmembrane domain is flanked on either side by the cell penetrating domain on one side with the ligand binding domain on the opposite side.

The attachment of the ligand binding domain to the transmembrane domain, and attachment of the transmembrane domain to the intracellular domain to produce the fusion peptides of the invention may be effected by any means which produces a link between the two constituents which is sufficiently stable to withstand the conditions used and which does not alter the function of either constituent. Preferably, the link between the constituents is a covalent bond. For example, recombinant techniques can be used to covalently attach the cell penetrating domain from the Tat protein to a hydrophobic transmembrane domain and extracellular domain, such as by joining the nucleic acid sequence encoding the extracellular domain with the nucleic acid encoding the transmembrane domain and Tat, and introducing the resulting nucleic acid construct into a cell capable of expressing the newly formed conjugate. The covalent linkage can be stable or labile to allow separation of a domain following insertion if desired.

The fusion peptides of the invention can be prepared using standard solid phase (or solution phase) peptide synthesis methods, as is known in the art. In addition, the DNA encoding these peptides can be synthesized using commercially available oligonucleotide synthesis instrumentation and produced recombinantly using standard recombinant production systems. Production using solid phase peptide synthesis is necessitated if non-gene-encoded amino acids or other chemical moieties are to be included.

As it is more generally known, a fusion peptide, polypeptide or protein is an expression product resulting from the fusion of two genes. Such a peptide can be produced, e.g., in recombinant DNA expression studies or, naturally, in certain viral oncogenes. As used herein, it more specifically refers to a peptide, polypeptide, or protein wherein a peptide comprising a cell penetrating domain is linked to a transmembrane domain.

The production of fusion proteins is well known to one skilled in the art (see, e.g., U.S. Pat. Nos. 5,908,756; 5,907,085; 5,906,819; 5,905,146; 5,895,813; 5,891,643; 5,891,628; 5,891,432; 5,889,169; 5,889,150; 5,888,981; 5,888,773; 5,886,150; 5,886,149; 5,885,833; 5,885,803; 5,885,779; 5,885,580; 5,883,124; 5,882,941; 5,882,894; 5,882,864; 5,879,917; 5,879,893; 5,876,972; 5,874,304; and 5,874,290). For a general review of the construction, properties, applications and problems associated with specific types of fusion molecules used in clinical and research medicine, see Chamow et al., 1999 Antibody Fusion Proteins, John Wiley.

Alternatively, the two separate nucleotide sequences can be expressed in a cell or can be synthesized chemically and subsequently joined, using techniques known to those of skill in the art. Alternatively, the fusion peptide can be synthesized chemically as a single amino acid sequence (i.e., one in which all constituents are present) and, thus, joining is not needed.

Numerous chemical cross-linking methods are known and potentially applicable for conjugating a cell penetrating domain of a protein or peptide to the transmembrane domain of a protein or peptide and for conjugating a transmembrane domain of a protein or peptide to a ligand binding domain of a protein or peptide. Many known chemical cross-linking methods are non-specific, i. e., they do not direct the point of coupling to any particular site on the cell penetrating domain, transmembrane domain or ligand binding domain. As a result, use of non-specific cross-linking agents can attack functional sites or sterically block active sites, rendering the conjugated proteins biologically inactive.

A preferred approach to increasing coupling specificity of one domain to another in the practice of this invention is direct chemical coupling of one domain to a functional group of another domain found only once or a few times in one or both of the polypeptides to be cross-linked. For example, in many proteins, cysteine, which is the only protein amino acid containing a thiol group, occurs only a few times. Also, for example, if a polypeptide contains no lysine residues, a cross-linking reagent specific for primary amines will be selective for the amino terminus of that polypeptide. Successful utilization of this approach to increase coupling specificity of one domain to another (e.g., a transmembrane domain of a protein to a cell penetrating domain of a protein) requires that the polypeptide have the suitably rare and reactive residues in areas of the molecule that can be altered without loss of the molecule's biological activity.

Cysteine residues can be replaced by other amino acids when they occur in a region of a polypeptide sequence where their participation in a cross-linking reaction would likely interfere with biological activity. When a cysteine residue is replaced by another amino acid, it is typically desirable to minimize resulting changes in polypeptide folding. Changes in polypeptide folding are minimized when the replacement amino acid is chemically and sterically similar to cysteine. For these reasons, serine is preferred as a replacement for cysteine.

A cysteine residue can also be introduced into a polypeptide's amino acid sequence for cross-linking purposes. When a cysteine residue is introduced, introduction at or near the amino or carboxy terminus is preferred. Conventional methods are available to accomplish such amino acid sequence modifications, whether the polypeptide of interest is produced by chemical synthesis or expression of recombinant DNA.

Coupling of the two constituents can be accomplished via a coupling or conjugating agent. There are several intermolecular cross-linking reagents which can be utilized (see, for example, Means et al., 1974, Chemical Modification of Proteins, 39-43, Holden-Day). Among these reagents are, for example, J-succinimidyl 3-(2-pyridyldithio)propionate (SPDP) or N,N′-(1,3-phenylene)bismaleimide (both of which are highly specific for sulfhydryl groups and form irreversible linkages); N,N′-ethylene-bis-(iodoacetamide) or other such reagent having six to eleven carbon methylene bridges (which are relatively specific for sulfhydryl groups); and 1,5-difluoro-2,4-dinitrobenzene (which forms irreversible linkages with amino and tyrosine groups). Other cross-linking reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Cross-linking reagents can be homobifunctional, i.e., having two functional groups that undergo the same reaction. A preferred homobifunctional cross-linking reagent is bismaleimidohexane (BMH). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of polypeptides that contain cysteine residues.

Cross-linking reagents can also be heterobifunctional. Heterobifunctional cross-linking agents have two different functional groups, for example an amine-reactive group and a thiol-reactive group, that will cross-link two proteins having free amines and thiols, respectively. Examples of heterobifunctional cross-linking agents are succinimidyl 4-(N-maleimidomethyl)cyclohexane-1-carboxylate (SMCC), m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Cross-linking reagents often have low solubility in water. A hydrophilic moiety, such as a sulfonate group, can be added to the cross-linking reagent to improve its water solubility. Sulfo-MBS and sulfo-SMCC are examples of cross-linking reagents modified for water solubility.

Many cross-linking reagents yield a conjugate that is essentially non-cleavable under cellular conditions. However, some cross-linking reagents contain a covalent bond, such as a disulfide, that is cleavable under cellular conditions. For example, dithio-bis(succinimidylpropionate) (DSP), Traut's reagent and N-succinimidyl 3-(2-pyridyldithio)propionate (SPDP) are well-known cleavable cross-linkers. The use of a cleavable cross-linking reagent permits the cargo moiety to separate from the transport polypeptide after delivery into the target cell. Direct disulfide linkage can also be useful.

Some new cross-linking reagents such as n-γ-maleimidobutyryloxy-succinimide ester (GMBS) and sulfo-GMBS, have reduced immunogenicity. In some embodiments of the present invention, such reduced immunogenicity can be advantageous.

Numerous cross-linking reagents, including the ones discussed above, are commercially available. Detailed instructions for their use are readily available from the commercial suppliers. A general reference on protein cross-linking and conjugate preparation is Wong, 1991 Chemistry of Protein Conjugation and Cross-Linking, CRC Press.

Chemical cross-linking can include the use of spacer arms. Spacer arms provide intramolecular flexibility or adjust intramolecular distances between conjugated moieties and thereby can help preserve biological activity. A spacer arm can be in the form of a polypeptide moiety comprising spacer amino acids. Alternatively, a spacer arm can be part of the cross-linking reagent, such as in “long-chain SPDP” (Pierce Chemical).

The invention should not be construed to include only the binding and cross-linking techniques described herein, but should also be construed to include other binding and cross-linking techniques not disclosed herein.

Fusion Peptides for Transducing Cells with Nucleic Acids and other Molecules

The present invention also relates to compositions and methods for transporting a molecule of interest such as a nucleic acid, into either the cytoplasm or nucleus of a cell using a fusion peptide. In its preferred form, the fusion peptide comprises two domains for translocating a nucleic acid into a cell, namely, a cell penetrating domain linked to an adapter or linker domain. The linker or adapter domain in turn is what links to the nucleic acid or molecule of interest. In one embodiment, the adapter domain can comprise basic, positively charge amino acids or other basic chemical moieties, including but not limited to those sequences and moieties presented in Table 3. The invention should not be construed to be limited solely to include only the sequences and moieties described herein, but should also be construed to include others as well.

The electrostatic interactions between the basic adapter domain and the acidic phosphate groups in the nucleic acid molecule are of sufficient strength to enable membrane transduction of fusion peptide-nucleic acid complexes without covalent linkage between the fusion peptide and nucleic acid.

TABLE 3

Adapter domains for nucleic acid binding

| | | | |
|---|---|---|---|
| Binding Moiety | Peptide Sequence | SEQ ID | Reference |

| | | | |
|---|---|---|---|
| Polylysine | (K)n | 28 | Zauner et al, 1998, Adv. Drug |
| | | | Deliv. Rev. 30:97-113 |
| Histidylated polylysine | HoK | 29 | Midoux et al., 1999, Bioconjug. |
| | | | Chem. 10:406-411 |
| (2-dimethylamino)ethyl | | | Cherng et al., 1996, Pharm. Res. |
| methacrylate | 13:1038-1042 | | |
| Polyallylamine | | | Boussif et al., 1999, Bioconjugate |
| | | | Chem. 10:877-883 |
| Polyethyleneimine | | | Boussif et al., 1995, Proc. Natl. |
| | | | Acad. Sci. USA 92:7297-7301 |
| 4₆ | LARLLARLLARLL | 30 | Niidome et al., 1997, J. Biol. |
| | ARLLARLLARL | | Chem. 272:15307-15312 |
| Protamine | | | Birchall et al., 2000, Int. J. Pharm. |
| | | | 197:221-231 |
| Branched peptide | (KKKK)₂KGGC | 31 | Plank et al., 1999, Human Gene |
| Branched peptide | (RRLRR)₂KGGC | 32 | Ther. 10:319-332 |
| Branched peptide | (RWRR)₂KGGC | 33 | |
| Hydroxyapatite | | | |
| Polyarginine | (R)n | 35 | |
| Mixed polyarginine | [(R)p(X)m]n | 55 | |
| copolymer (X is any amino | | | |
| acid) | | | |
| Mixed polylysine | [(K)p(X)m]n | 56 | |
| copolymer (X is any amino | | | |
| acid) | | | |
| glucaramide polymers | | | Goldman et al., 1997, Nat. |
| | | | Biotechnol, 15:462-466 |
| N-substituted glycine | | | Murphy et al., 1998, Proc. Natl. |
| (NSG) peptoids | | | Acad. Sci. USA 95:1517-1522 |
| Polyamines | | | Behr et al., 1989, Proc. Natl. Acad. |
| | | | Sci. USA 86:6982-6986 |

Uses of Fusion Peptides for Transducing Nucleic Acids and Other Molecules across Cell Membranes

The fusion peptides of the invention may be used to translocate nucleic acids across the cell membrane into the cytoplasm, and if desired, subsequent translocation to the nucleus. The nuclear localization of the nucleic acids enables the expression of the genes encoded by the nucleic acids. The present invention thereby provides an improved method for delivering nucleic acids to the nuclei of cells, in particular, mammalian cells, e.g., exogenous DNA for transforming human cells. This method for example generally comprises providing to the cell targeted for transformation a specifically designed fusion peptide linked to a specific nucleic acid comprising the exogenous DNA desired to be targeted to the nucleus and expressed in the transformant.

In one embodiment of the invention the translocated nucleic acid is an oligonucleotide. In another embodiment, the translocated nucleic acid is double stranded DNA. In yet another embodiment the nucleic acid can be RNA or a ribozyme.

In one embodiment of the invention, the nucleic acid which is translocated is an oligomer, preferably from 5 to 25 nucleotides in length. In yet another embodiment, the oligomer is from 25 to 100 nucleotides in length. In a further embodiment, the oligomer is greater than 100 nucleotides in length. In another embodiment the nucleic acid is double stranded DNA which is less than 400 base pairs in length. In yet another embodiment of the invention, the DNA is 400 base pairs or greater in length.

In certain embodiments, the DNA which is translocated across the cell membrane by a fusion peptide of the invention is a plasmid.

In yet another embodiment, the nucleic acid which is translocated across the cell membrane by a fusion peptide is single stranded RNA, double stranded RNA, ribozymes, or other RNA structures.

The invention should not be construed to include only the nucleic acids or molecules described herein which are capable of being translocated by a fusion peptide across a cell membrane. The invention should also be construed to include other types of molecules and nucleic acids which can be transported, or whose transport can be facilitated, by fusion peptides.

According to the invention, the fusion peptide linked to the exogenous nucleic acid, such as, a cell penetrating domain linked to a DNA sequence encoding a therapeutic gene, can be delivered to the cells via uptake of the cell penetrating domain but can also be enhanced by means such as, but not restricted to, electroporation, microinjection, induced uptake, microprojectile bombardment, liposomes, viral vectors or other means as are known in the art. Accordingly, the present invention provides novel means for the in vivo and in vitro translocation and integration of exogenous nucleic acids desired to be expressed within hosts or host cells, particularly for the purpose of gene therapy.

In the course of the experiments which led to the present invention it was found that DNA and oligonucleotides could be efficiently translocated into cells by using a fusion peptide which can link to a nucleic acid. The link is via an adapter domain on the peptide. In addition to the sequences and chemical moieties set forth in Table 3, the adapter domain of the fusion peptide can be selected from the group consisting of one or more of intercalating agents, cross-linking reagents, incorporating molecules, ionically and hydrophobically interacting molecules. The invention should not be construed to be limited solely to the adapter domain sequences and moieties of Table 3, but should instead be construed to include other adapter domain sequences and moieties as well.

A cell penetrating domain of the fusion polypeptide can be linked to a nucleic acid directly via a covalent, ionic or hydrophobic interaction. In the alternative, it can be indirectly linked to a nucleic acid with a spacer or adapter domain being positioned between the cell penetrating domain and the nucleic acid.

In one embodiment, the nucleic acid which is transported, is transported across a eukaryotic cell membrane. Eukaryotic cells should be construed to include mammalian cells, fish cells, frog cells, and nematode cells, but should not be construed to include only the cells described herein. In a preferred embodiment the cells are human cells. Based on the data disclosed herein, it is likely that the invention also includes prokaryotic cells.

Nucleic Acids Encoding Fusion Peptides

The present invention further provides isolated nucleic acid molecules which encode the peptides of the invention and conservative nucleotide substitutions thereof, preferably in isolated form. Conservative nucleotide substitutions include nucleotide substitutions which do not cause the substitution of a particular amino acid for another, as most amino acids have more than one codon (see King and Stansfield (Editors), A Dictionary of Genetics, Oxford University Press, 1997). Conservative nucleotide substitutions therefore also include silent mutations and differential codon usage. The invention includes nucleic acids encoding the peptides set forth in SEQ ID NOs: 36, 37, 38, 39, 41 and 42 and conservative nucleotide substitutions thereof (see Examples). Any nucleic acid that encodes the peptides set forth in SEQ ID NOs: 36, 37, 38, 39, 41 and 42 is encompassed in the invention, given the multiple permutations of nucleotide sequences possible which encode these peptides.

One skilled in the art would appreciate, based upon the disclosure provided herein, that modified nucleic acid sequences, i.e. nucleic acids having sequences that differ from the gene sequences encoding the naturally-occurring protein, are also encompassed by the invention, so long as the modified nucleic acid still encodes a protein or peptide that allows proper function of the fusion peptide. These modified gene or nucleic acid sequences include modifications caused by point mutations, modifications due to the degeneracy of the genetic code or naturally occurring allelic variants, and further modifications that have been introduced by genetic engineering, i.e., by the hand of man.

Techniques for introducing changes in nucleotide sequences that are designed to alter the functional properties of the encoded proteins or polypeptides are well known in the art. Such modifications include the deletion, insertion, or substitution of bases, and thus, changes in the amino acid sequence. Changes may be made to increase the activity of a protein, to increase its biological stability or half-life, to change its glycosylation pattern, and the like. All such modifications to the nucleotide sequences encoding such proteins are encompassed by this invention.

The invention should not be construed to include only the nucleic acids encoding proteins or peptides which are described herein. The invention should be construed to include other nucleic acids encoding other peptides, as well as modifications to the nucleic acids described herein. Modifications to the primary structure of the nucleic acid itself by deletion, addition, or alteration of the amino acids incorporated into the protein sequence during translation can be made without destroying the activity of the peptide. Such substitutions or other alterations result in a peptide having an amino acid sequence encoded by a nucleic acid falling within the contemplated scope of the present invention.

It should be construed that the domains of a fusion peptide encoded by a nucleic acid of the invention may be in various orientations, and it should not be construed that a cell penetrating domain and a transmembrane domain encoded by a nucleic acid must be in a particular orientation. That is, in one embodiment a cell penetrating domain may be 5′ to a transmembrane domain and in another embodiment a cell penetrating domain may be 3′ to a transmembrane domain. One embodiment may be preferable to another depending on whether a fusion peptide will be produced from a nucleic acid encoding it while outside the cell or whether a nucleic acid encoding a fusion peptide is transduced into a cell and a fusion peptide is then produced inside the cell. The orientation of the domains of a fusion peptide can thus be manipulated to select the manner in which the fusion peptides orients itself with respect to the inside or outside of the cell membrane.

Using Antibodies as Probes or Regulators of Fusion Peptides

The invention also includes a method by which antibodies can be generated and used as probes to detect the fusion peptides of the invention as well as to modulate their function. The preparation and use of antibodies as probes or to modulate function is a technique known by those skilled in the art. The generation of polyclonal antibodies is accomplished by inoculating the desired animal with the antigen and isolating antibodies which specifically bind the antigen therefrom.

Antibody probes are prepared by immunizing suitable mammalian hosts in appropriate immunization protocols using the peptides. Peptides or proteins comprising the extracellular or intracellular domains are generally of sufficient length, or if desired, as required to enhance immunogenicity, can be conjugated to suitable carriers. Methods for preparing immunogenic conjugates with carriers such as BSA, KLH, or other carrier proteins are well known in the art. In some circumstances, direct conjugation using, for example, carbodiimide reagents can be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies can be prepared using the standard method of Kohler et al., 1992, Biotechnology 24:524-526 or modifications which effect immortalization of lymphocytes or spleen cells, as is generally known. The immortalized cell lines secreting the desired antibodies are screened by immunoassay in which the antigen is the peptide hapten, peptide or protein. When the appropriate immortalized cell culture secreting the desired antibody is identified, the cells can be cultured either in vitro or by production in ascites fluid.

The desired monoclonal antibodies can be recovered from the culture supernatant or from the ascites supernatant. Fragments of the monoclonals or the polyclonal antisera, which contain the immunologically significant portion, can be used as antagonists, as well as the intact antibodies. Use of immunologically reactive fragments, such as the Fab or Fab′ fragments of F(ab)₂ are often preferable, especially in a therapeutic context, as these fragments are generally less immunogenic than the whole immunoglobulin.

The antibodies or fragments can also be produced, using current technology, by recombinant means. Antibody regions that bind specifically to the desired regions of the protein can also be produced in the context of chimeras with multiple species of origin.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be “rationally selected or designed” when the agent is chosen on a non-random basis which takes into account the sequence of the target site and/or its conformation in connection with the agent's action. The agents of the present invention include, but are not limited to, for example, peptides, small molecules, vitamin derivatives, as well as carbohydrates. A skilled artisan can readily recognize that there is no limit as to the structural nature of the agents of the present invention.

Monoclonal antibodies can be used effectively intracellularly to avoid uptake problems by cloning the gene and then transfecting the gene encoding the antibody. Such a nucleic acid encoding the monoclonal antibody gene obtained using the procedures described herein can be cloned and sequenced using technology which is available in the art.

Monoclonal antibodies directed against full length or peptide fragments of a protein or peptide can be prepared using any well known monoclonal antibody preparation procedure. Quantities of the desired peptide can also be synthesized using chemical synthesis technology. Alternatively, DNA encoding the desired peptide can be cloned and expressed from an appropriate promoter sequence in cells suitable for the generation of large quantities of peptide. Monoclonal antibodies directed against the peptide are generated from mice immunized with the peptide using standard procedures as referenced herein. A nucleic acid encoding the monoclonal antibody obtained using the procedures described herein can be cloned and sequenced using technology which is available in the art. Further, the antibody of the invention can be “humanized” using the existing technology known in the art.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. 1989, Molecular Cloning: A Laboratory Manual, Cold Spring Harbor, N.Y.).

Bacteriophage which encode the desired antibody, can be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA can be generated following previously described procedures (Marks et al., 1991, J. Mol. Biol. 222:581-597). Panning of phage so generated for the isolation of a desired antibody is conducted in a manner similar to that described for phage libraries comprising Fab DNA. The invention should also be construed to include synthetic phage display libraries in which the heavy and light chain variable regions can be synthesized such that they include nearly all possible specificities (Barbas, 1995, Nature Medicine 1:837-839; de Kruif et al. 1995, J. Mol. Biol. 248:97-105).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The invention should not be construed to be limited to only the antibodies, methods for making antibodies, or uses of antibodies, described herein, but should be construed to include others as well.

Methods of Treating Diseases

The invention also relates to methods of inhibiting or treating diseases or disorders. Some examples of diseases which can be treated according to the methods of the invention are described herein. These diseases or disorders include, but are not limited to, those which could benefit from the addition of surface receptors to cells. In one embodiment, these diseases or disorders include those wherein there are ligand-receptor binding interaction problems due to lack of a receptor. In yet another embodiment, the disease or disorder is due to a defective receptor. Examples of these types of diseases include, but are not limited to cancers, various proliferative disorders such as psoriasis, and cystic fibrosis. Many diseases and disorders would also benefit from the methods of the invention in which fusion peptides are used to transport or facilitate transduction of nucleic acids or other molecules into cells.

The invention should not be construed as being limited solely to these examples, as other diseases which are at present unknown, once known, may also be treatable using the methods of the invention. In one aspect the treated disease is cancer. A cancer may belong to any of a group of cancers which have been described, as well as any other cancers not yet described.

It will be recognized by one of skill in the art that the various embodiments of the invention as described above relating to methods of treating diseases encompasses diseases not described herein. Thus, it should not be construed that embodiments for diseases described herein do not apply to other diseases.

Kits for Administering Fusion Peptides

The method of the invention includes a kit comprising a composite receptor fusion peptide identified in the invention and an instructional material which describes administering the fusion peptide to a cell or to an animal. Preferably the animal is a human. The invention should be construed to include other embodiments of kits that are known to those skilled in the art, such as a kit comprising a (preferably sterile) solvent suitable for dissolving or suspending the composition of the invention prior to administering the compound to a cell or an animal. The kit should not be construed to include only the materials described herein, but should be construed to include other fusion peptides, compositions, and other molecules as well.

In another embodiment, the method of the invention includes a kit comprising a fusion peptide comprising at least one cell penetrating domain which is linked to an adapter domain capable of binding to a molecule. Preferably the molecule is a nucleic acid. The kit further comprises an instructional material, which describes administering the fusion peptide to a cell or to an animal. Preferably the animal is a human. The invention should be construed to include other embodiments of kits that are known to those skilled in the art, such as a kit comprising a (preferably sterile) solvent suitable for dissolving or suspending the composition of the invention prior to administering the compound to a cell or an animal. The kit should not be construed to include only the materials described herein, but should be construed to include other fusion peptides, compositions, and other molecules as well.

Administration of Fusion Peptides

The invention further relates to the administration of an identified compound in a pharmaceutical composition to practice the methods of the invention, the composition comprising the compound or an appropriate derivative or fragment of the compound and a pharmaceutically-acceptable carrier.

In one embodiment, the pharmaceutical compositions useful for practicing the invention can be administered to deliver a dose of between 1 ng/kg/day and 100 mg/kg/day.

Other pharmaceutically acceptable carriers which are useful include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer, for example, fusion peptides, fragments, or derivatives, and/or a nucleic acid encoding the same according to the methods of the invention. The method should not be construed to be limited to a fusion peptide comprising a composite receptor or a fusion peptide comprising an adapter domain bound to a nucleic acid, but should be construed to include other fusion proteins or peptides, fragments or derivatives thereof, as well as other types of molecules, agents, or compounds which have composite receptor activity or molecule transport activity.

Compounds which are identified using any of the methods described herein may be formulated and administered to a mammal for treatment of various viral related diseases described herein.

The invention encompasses the preparation and use of pharmaceutical compositions comprising a compound useful for treatment of various viral related diseases described herein. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

It will be recognized by one of skill in the art that the fusion peptides, molecules, and compositions of the invention can be administered in vitro to cells or tissues as part of an ex vivo therapy or use for cells or tissues that will then be returned to the subject.

Biochemical, Molecular Biology, Microbiology and Recombinant DNA Techniques

In accordance with the present invention, as described above or as discussed in the Examples below, there can be employed conventional biochemical, molecular biology, microbiology and recombinant DNA techniques which are known to those of skill in the art. Such techniques are explained fully in the literature. See for example, Sambrook et al., 1989 Molecular Cloning—a Laboratory Manual, Cold Spring Harbor Press; Glover, (1985) DNA Cloning: a Practical Approach; Gait, (1984) Oligonucleotide Synthesis; Harlow et al., 1988 Antibodies—a Laboratory Manual, Cold Spring Harbor Press; Roe et al., 1996 DNA Isolation and Sequencing: Essential Techniques, John Wiley; and Ausubel et al., 1995 Current Protocols in Molecular Biology, Greene Publishing.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### Composite Receptor Expression on the Cell Membrane Surface

There is a need in the art to be able to easily add surface receptors to cell membranes without using gene therapy techniques. The ability to quickly and easily add specific receptors to the surface of a cell using fusion peptides is disclosed in the present invention.

The Materials and Methods used in the present example are now described.

Fusion Peptides

For this example, the Tat-K₁₆ peptide component of the fusion peptide is a fluorescein (fluor) labeled thirty-five residue polypeptide (fluor-G₄YGRKKRRQR₃G₄K₁₆) (SEQ ID NO: 36) while the Tat peptide is a fluorescein labeled thirteen residue polypeptide (fluor-G₄YGRKKRRQR) (SEQ ID NO: 37) where YGRKKRRQR (SEQ ID NO: 21) is the Tat cell penetrating translocation domain.

The fusion peptide Composite receptor-1 (CR1) comprises a transmembrane domain, the sequence of which forms a stable alpha helix in aqueous solutions and readily anchors in a lipid bilayer (Percot et al., 1999, Biopolymers 50:647-655). The specific structure of fluorescein (fluor) labeled CR1 was fluor-G₃YGRKKRRQR₃G₃AAL₃A₁₉WK₈-biotin (SEQ ID NO: 38) where G₃YGRKKRRQR₃G₃ (SEQ ID NO: 43) is the intracellular domain, YGRKKRRQR (SEQ ID NO: 21) is the cell penetrating translocation domain, AAL₃A₁₉W (SEQ ID NO: 22) is the transmembrane domain and K₈-biotin (SEQ ID NO: 34) is the extracellular domain.

The fusion peptide comprising fluorescein labeled composite receptor-2 (CR2) employed the following sequence: fluor-G₄YGRKKRRQR₃G₃MPNLWFL₂FLGLVA₂MQL₅FL₃F₂LVYW DHFECSCTGLPF (SEQ ID NO: 39) where G₄YGRKKRRQR₃G₃MP (SEQ ID NO: 44is the intracellular domain, YGRKKRRQR (SEQ ID NO: 21) is the cell penetrating domain, LWFLLFLGLVAAMQL₅FL₃FFLVY (SEQ ID NO: 45) is the transmembrane domain and WDHFECSCTGLPF (SEQ ID NO: 46) is the extracellular domain.

The fusion peptide comprising composite receptor-1R (CR1R) employed the following sequence: biotin-G₃YGRKKRRQR₃G₃AAL₃A₁₉WK₈-FITC (SEQ ID NO: 51). The fusion peptides designated CR1 and CR1R have the same amino acid structure, but contain their lateral ligands in opposite position.

G3 or G4 domains are used as flexible linkers to connect functional domains in fusion peptides.

Preparation of Fusion Peptides: Synthesis and Purification of Peptides

Peptides for this and the other examples were prepared using Fmoc solid phase peptide synthesis techniques. The peptides were then purified by HPLC using a C18 reverse phase column.

Tat-K₁₆ was dissolved at a concentration of 8 mM in Hepes buffer (pH 7.5). CR1 was dissolved at a concentration of 800 μM in 0.5 M Hepes buffer (pH 7.5). CR2 was dissolved at a concentration of 80 μM in 80% DMSO, 100 mM Hepes buffer (pH 7.5) and 50 mM DTT. All peptides were diluted with DPBS to the appropriate concentrations before the experiment. Peptides were administered to the cells in concentrations ranging from 0-800 μM.

Cell Culture

SK-BR-3 human adenocarcinoma cells (ATCC No. HTB-30) were grown in A-medium (McCoy 5A medium supplemented with 15% fetal bovine serum, glutamine, penicillin and streptomycin), harvested while in the exponential phase of growth, washed in Dulbecco's phosphate buffered saline (DPBS), and then resuspended in DPBS or DPBS containing 0.5% Bovine Serum Albumin (DPBS-BSA).

Administration of Composite Receptor Peptides

Tat-K₁₆ and Tat peptides were each dissolved at a concentration of 8 mM in Hepes buffer (pH 7.5). CR1 and delta CR1 (SEQ ID NO: 57) were each dissolved at a concentration of 800 μM in 0.5 M Hepes buffer (pH 7.5). CR2 was dissolved at a concentration of 80 μM in 80% DMSO, 100 mM Hepes buffer (pH 7.5) and 50 mM DTT. All peptides were diluted with DPBS to the appropriate concentrations before the experiment. SK-BR-3 cells were collected in the exponential phase of growth, washed, and resuspended in DPBS at a concentration of 10⁶ cells per ml, incubated with 800 nM CR1 or 400 nM CR2 or 800 nM Tat-K₁₆ for fifteen minutes at 37° C., then washed with DPBS.

Microscopy and Fluorescence Analysis

Fluorescent images were captured using commercial software (Perceptive Scientific Imaging, League City, Tex.), a Sensys Camera (Photometrix, Tucson, Ariz.), and an Olympus microscope with multiple excitation and emission filters (Chroma Technology, Battleboro, Vt.). The nuclear DNA was counter-stained by mounting in Vectashield (Vector Laboratories, Burlingame, Calif.) containing 2 μg/ml DAPI (4′,6 diamidino-2-phenylindole).

Other methods which were used but not described herein are well known and within the competence of one of ordinary skill in the art of cellular and molecular biology.

The Results of the experiments described in this example are now presented.

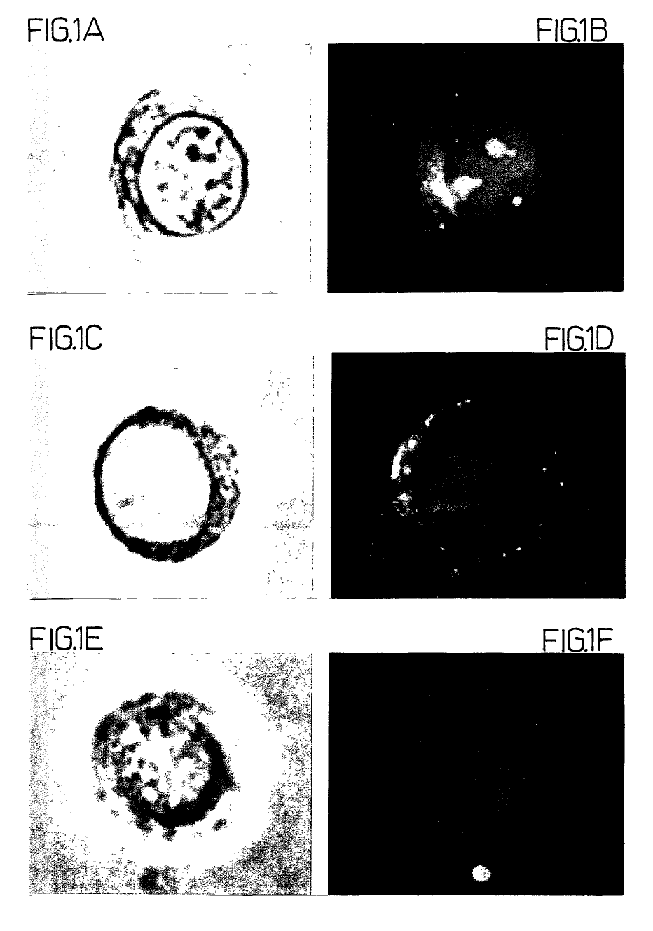

FIG. 1, comprising FIG. 1A, FIG. 1B, FIG. 1C, FIG. 1D, FIG. 1E, and FIG. 1F, is a series of images of photomicrographs depicting that the composite receptors (CR) CR1 and CR2 are localized at the cell surface, while Tat-K₁₆ peptide localizes in the cytoplasm and the nucleus. SK-BR-3 cells were collected and washed and resuspended in DPBS. 5×10⁴ cells were mixed with 800 nM TAK16, 800 nM CR1 or 400 nM CR2 in 40 μl DPBS, incubated 15 minutes at room temperature, washed in DPBS-BSA, then resuspended in 50 μl DPBS-BSA and washed and applied to slides. The nuclear DNA was counter-stained by mounting in Vectashield containing 2 μg/ml DAPI. Fluorescence was analyzed by fluorescence microscopically using a monochrome filter for FITC.

The images on the left represent phase contrast photomicrographs and those on the right represent fluorescent photomicrographs. SK-BR-3 cells were incubated with FLU-TAK16 (FIG. 1A and FIG. 1B), CR1 (FIG. 1C and FIG. 1D), or CR2 (FIG. 1E and FIG. 1F), The peptides (Flu-TAK16, CR1 and CR2) are labeled with fluorescein and appear green (darker), while the nuclei are stained with DAPI and appear blue (darker than surrounding area). The borders of the cytoplasm are defined in the phase contrast images on the left. The composite receptor molecules CR1 and CR2, which contain both the cell penetrating and transmembrane domains, are localized at the periphery of SK-BR-3 cells, while the Tat-K₁₆ peptide is located throughout the cell. The CR1 peptide was observed to be localized at the periphery of the cells, in a speckled distribution. The CR2 peptide was also localized at the periphery of cells, but contrary to the more even distribution of CR1 at the cell surface, CR2 was localized in a few foci at the cell surface. Thus, it was concluded that neither CR1 nor CR2 were translocated through the cell membrane under these experimental conditions and that both localized at the cell surface.

FIG. 2 is a schematic illustration of various composite receptor structure variations. The variations included, but are not limited to, those described herein. In its most basic form, the composite receptor comprises a transmembrane domain and a cell penetrating domain, as depicted in FIG. 2A. In another form, the composite receptor further comprises a ligand binding domain in the extracellular domain of the composite receptor (FIG. 2B). Alternative forms can exist. For example, the ligand binding domain can be in the intracellular domain between the transmembrane domain and the cell penetrating domain as illustrated in FIG. 2C. Alternatively, the ligand binding domain can be on the end of the fusion molecule adjacent to the cell penetrating domain (FIG. 2D). The composite receptor can also comprise more than one ligand binding domain in various configurations as shown in FIG. 2E. In the drawing, the abbreviations TM, CPD, and LBD, stand for Transmembrane Domain, Cell Penetrating Domain, and Ligand Binding Domain, respectively.

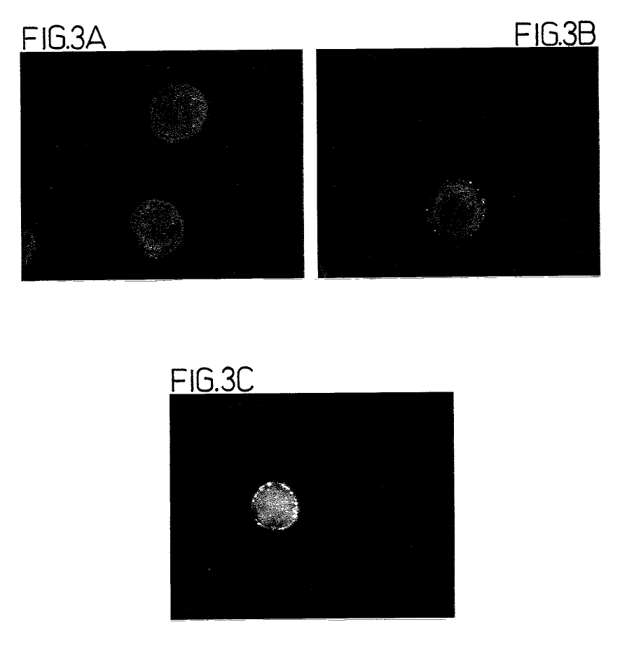

Next, FIG. 3, is a series of images of fluorescent micrographs illustrating the effects of a cell penetrating domain on the efficiency of composite receptor binding. SK-BR-3 cells were incubated with the fusion peptides CR1 (biotin-G₃YGRKKRRQR₃G₃AAL₃A₁₉WK₈-FITC; SEQ ID NO: 38) or delta-CR1 (FITC-G₃G₃AAL₃A₁₉WK₈-biotin; SEQ ID NO: 57) which have the same amino acid structure, but delta-CR1 has a deletion of the cell penetrating domain.

SK-BR-3 cells were collected and washed and resuspended in DPBS-BSA. Then, 5×10⁴ cells were mixed with 200 nM CR1 or delta-CR1 in 40 μl DPBS-BSA and incubated 15 minutes on ice. The cell were then washed in DPBS-BSA, resuspended in 50 μl DPBS-BSA, washed, and applied to slides. Fluorescence was analyzed by fluorescence microscopy using a monochrome filter for FITC. FIG. 3A depicts cells which received no peptides, thus, no fluorescence appears. FIG. 3B depicts cells incubated with 200 nM delta-CR1 and it can be seen that there is a low level of fluorescence on the cell membrane. FIG. 3C depicts cells incubated with 200 μM CR1.

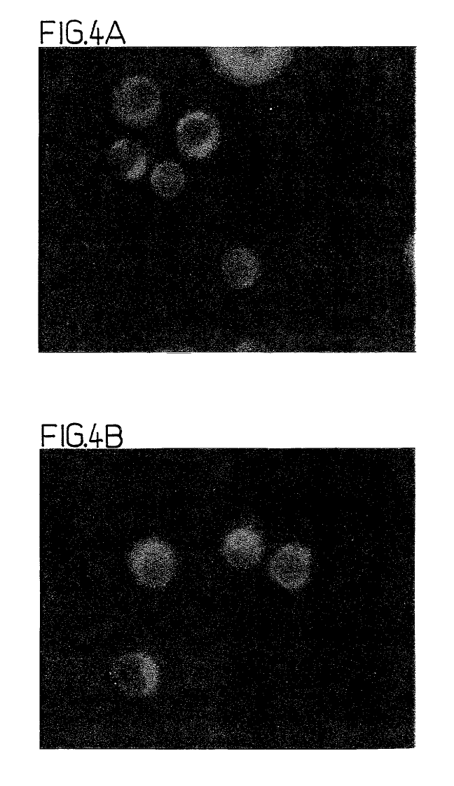

To demonstrate the next series of experiments, FIG. 4 is a series of images of fluorescent micrographs of SK-BR-3 cells incubated with the composite receptors CR1R (biotin-G₃YGRKKRRQR₃G₃AAL₃A₁₉WK₈-FITC; SEQ ID NO: 51) (FIG. 4A) or CR1 (FITC-G₃YGRKKRRQR₃G₃AAL₃A₁₉WK₈-biotin; SEQ ID NO: 38) (FIG. 4B) which have the same amino acid structure but different orientations of their lateral FITC groups. This difference in orientation can be detected using an anti-FITC antibody which cannot penetrate the cellular membrane and therefore can only bind FITC which is on the outside cell surface and not inside the cell. CR1 (FITC-G₃YGRKKRRQR₃G₃AAL₃A₁₉WK₈-biotin) contains FITC as an intracellular domain, while CR1R (biotin-G₃YGRKKRRQR₃G₃AAL₃A₁₉WK₈-FITC) contains FITC as its extracellular domain. For these experiments Alexa-Fluor 594 Rabbit polyclonal anti-FITC antibody (Molecular Probes) was used for detection of receptor orientation in the cellular membrane. SK-BR-3 cells were collected and washed in DPBS with 0.5% BSA (DPBS-BSA), resuspended in DPBS-BSA with 10 mM Sodium Azide and after a 20 minute incubation at room temperature were placed on ice. Then, 5×10⁴ cells were mixed with 60 nM CR1 or CR1R in 20 μl DPBS-BSA, incubated 30 minutes on ice, washed in DPBS-BSA-Azide, and resuspended in 50 μl DPBS-BSA-Azide. Then, 50 μl of anti-antibody in DPBS-BSA-Azide (dilution 1:50) was added, the suspension was incubated on ice 30 minutes, and then washed and applied to slides. Alexa-Fluor 594 fluorescence was analyzed using a fluorescent microscope and a monochrome filter for Cy3.5. It can be seen that the fluorescence for anti-fluorescein antibodies was higher for the CR1R peptide, which has the extracellular orientation of the FITC group.

Together, these results demonstrate the efficacy of the composite receptors of the present invention, and the ease and flexibility of their use.

### Example 2

### Translocation of Peptides Containing a Cell Penetrating Domain

In order for a fusion peptide to be able to transport a molecule such as a nucleic acid into a cell, the peptide needs to be able to penetrate the cell membrane. In this example, it is shown that fusion peptides of the invention can penetrate the cell membrane.

The Materials and Methods used in the present example are now described.

Fusion Peptides

For this example, the Tat-K₁₆ peptide is a fluorescein (fluor) labeled thirty-five residue polypeptide (fluor-G₄YGRKKRRQR₃G₄K₁₆) (SEQ ID NO: 36) while the Tat peptide is a fluorescein labeled thirteen residue polypeptide (fluor-G₄YGRKKRRQR) (SEQ ID NO: 37), where YGRKKRRQR (SEQ ID NO: 21) is the Tat cell penetrating translocation domain.

Cell Culture

SK-BR-3 human adenocarcinoma cells (ATCC No. HTB-30) were grown in A-medium (McCoy 5A medium supplemented with 15% fetal bovine serum, glutamine, penicillin and streptomycin), collected while in the exponential phase of growth, washed in Dulbecco's phosphate buffered saline (DPBS) and resuspended in DPBS at a concentration of 1×10⁶ cells/ml. Ten μl of cell suspension (10⁴ cells) was mixed with 90 μl DPBS containing peptide (final peptide concentration, 800 nM).

Exposure of Cells to Peptides

For use with cells, the fluor labeled peptides were first dissolved in 0.5 M Hepes buffer (pH 7.5) and then diluted in 10 mM Hepes buffer. Cells were incubated with the peptide for fifteen minutes at 37° C., washed two times with DPBS, resuspended in 100 μl DPBS, and then analyzed for the presence of fluorescent label.

Fluorescence Analysis

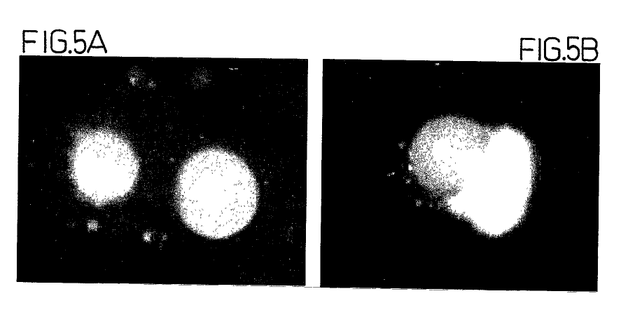

The nuclear DNA was counter-stained by mounting cells in Vectashield® containing 2 μg/ml DAPI (Vector Laboratories, Burlingame, Calif.). Treated cells were subjected to fluorescence microscopy. The images of the cells shown in FIG. 5 were obtained using an epifluorescence microscope. DNA counterstain is shown in blue and the peptide-fluorescein isothiocyanate (peptide-FITC) label is shown in green.

Other methods which were used but not described herein are well known and within the competence of one of ordinary skill in the art of cellular and molecular biology.

The Results of the experiments described in this example are now presented.

Human SK-BR-3 adenocarcinoma cells were incubated with fluorescein-labeled Tat or Tat-K₁₆. The results demonstrate that both Tat (FIG. 5A) and Tat-K₁₆ (FIG. 5B) peptides entered the cell and were located in both the cytoplasm and the nucleus of SK-BR-3 cells. These experimental results demonstrate that the Tat and Tat-K₁₆ peptides enter the cell and that they can become localized in both the cytoplasm and nucleus.

### Example 3

### Facilitation of Translocation of Molecules Across Cell Membranes by a Fusion Peptide

Translocation of Oligonucleotides with a Fusion Peptide: Tat-K₁₆ But Not Tat Peptide Facilitates Oligonucleotide Translocation Through the Cell Membrane

Oligonucleotides are examples of the types of macromolecules which can be transported using the techniques disclosed in the present invention. The Tat-K₁₆ peptide comprises an adapter domain consisting of 16 lysine residues, which domain is useful for binding nucleic acids (see Table 3 for a list of adapter domains). The Tat peptide does not have such a domain. The experiments performed herein demonstrate that the addition of an adapter domain to a peptide gives it the ability to transport a macromolecule across the cell membrane. Thus, in the course of the experiments described herein, it was discovered that a nucleic acid can be efficiently translocated into a cell using a fusion peptide with an adapter domain which can link to said nucleic acid.

The Materials and Methods used in the present example are now described.

Peptides and Oligonucleotides

For studies on the translocation of oligonucleotides, the oligonucleotides used included Cy3-O18 (5′-Cy3-TATATGATGGTACCGCAG-‘3-dT-5’) (dT_{N} bipolar oligonucleotide with inversion) (SEQ ID NO: 40), fluor-O-24 (fluor-TCAGAACTCACCTGTTAGACGCCA-3′-3-hydroxy-1-propyl; SEQ ID NO: 52), O-83-Fluor (TTGATAAGAGGTCATTTTTGCGGATGGCTTAGAGCTTAATTGCTGAATCT GGTGCTGTAGCTCAACATGTTTTAAATATGCAA-3′-fluor; SEQ ID NO: 53), and Bio-O-22 (biotin-CCAGACTGAGTATCTCCTATCA; SEQ ID NO: 54). The Tat-K₁₆ (fluor-G₄YGRKKRRQR₃G₄K₁₆; SEQ ID NO: 41) and Tat flour-G₄YGRKKRRQR; SEQ ID NO: 42) peptides were mixed with the oligonucleotide and then incubated as described below.

Peptide and Oligonucleotide Treatment of SK-BR-3 Cells

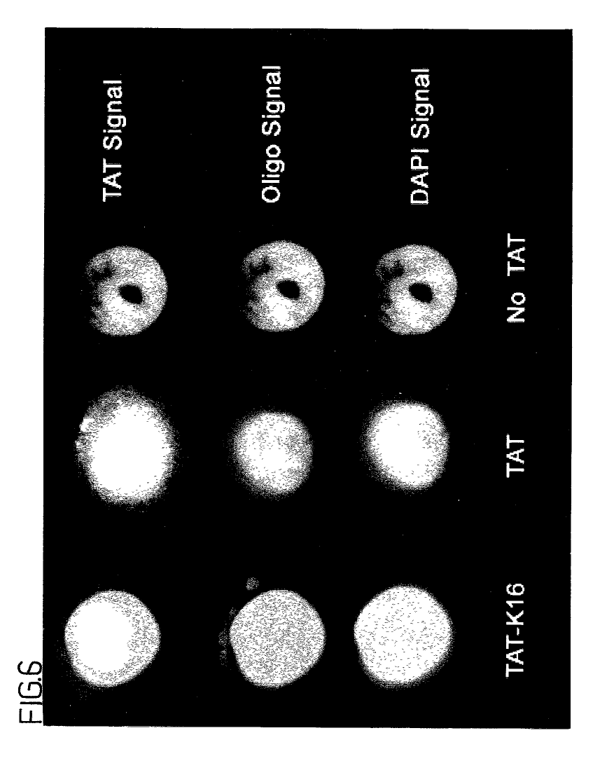

SK-BR-3 human adenocarcinoma cells were harvested, washed in DPBS, and then resuspended in DPBS at a concentration of 10⁶ cells/ml. Ten μl of the cell suspension (10⁴ cells) was resuspended in 100 μl DPBS containing 500 nM oligonucleotide and 80 nM peptide. Cells were incubated for fifteen minutes at 37° C., washed two times with 1.5 ml DPBS and resuspended in 100 μl DPBS. Nuclear DNA was counter-stained by mounting in Vectashield® containing 2 μg/ml DAPI. The images of the cells shown in FIG. 6 were obtained using an epifluorescence microscope (DNA counterstain shown in blue; the Tat-FITC label shown in green, oligonucleotide-Cy3 shown in red). Cy3 is a CyDye fluorescent dye used for fluorescent labeling of molecules (Amersham Pharmacia, Piscataway, N.J.).

Flow Cytometry

Flow cytometric analyses of cell subpopulations for these and other experiments described herein were performed using a FACS Vantage flow cytometer (Becton-Dickinson Immunocytometry Systems, San Jose, Calif.). The cells were excited at 488 nm. The FITC fluorescence was collected through a 530/30 nm band pass filter. A minimum of ten thousand cells was interrogated in each sample. Analysis of data was performed using CellQuest software (Becton-Dickinson).

To detect fluorescent labeled molecules associated with cells, the cells were first harvested in DPBS, incubated with a fluorescent labeled molecule of interest at varied concentrations (e.g., oligonucleotide at 50 μM mixed with 1×10⁵ cells in 10 μl DPBS), incubated for 15 minutes at room temperature, diluted with 200 μl growth medium, and then analyzed by fluorescence activated cell sorting (FACS).

Other methods which were used but not described herein are well known and within the competence of one of ordinary skill in the art of cellular and molecular biology.

Results

The Results of the experiments described in this example are now presented.

First, it was determined whether a Tat peptide without an adapter domain could facilitate translocation of an oligonucleotide across a cell membrane. The results demonstrate that the Tat-K₁₆ peptide, which includes a 16 residue lysine adapter domain, but not Tat peptide (which has no adapter domain), facilitated oligonucleotide Cy3-O18 (5′-Cy3-TATATGATGGTACCGCAG-′3-dT-5′) (dT_{N} bipolar oligonucleotide with inversion) (SEQ ID NO: 40) translocation through the cell membrane (FIG. 6). Fluorescence analyses of cells treated with both the Flu-TAK₁₆ and oligonucleotide showed that Cy3 label (red) of the oligonucleotide was located in the cytoplasm as well as in the nucleus. Furthermore, the Cy3 (oligonucleotide) and FITC (Tat-K₁₆ peptide) signals did not necessarily co-localize within a cell. The Cy3 label was not detected in cells incubated in the presence of a mixture of Cy3-oligonucleotide and Flu-TAT, nor was it detected in cells incubated in the presence of Cy3-oligonucleotide alone.

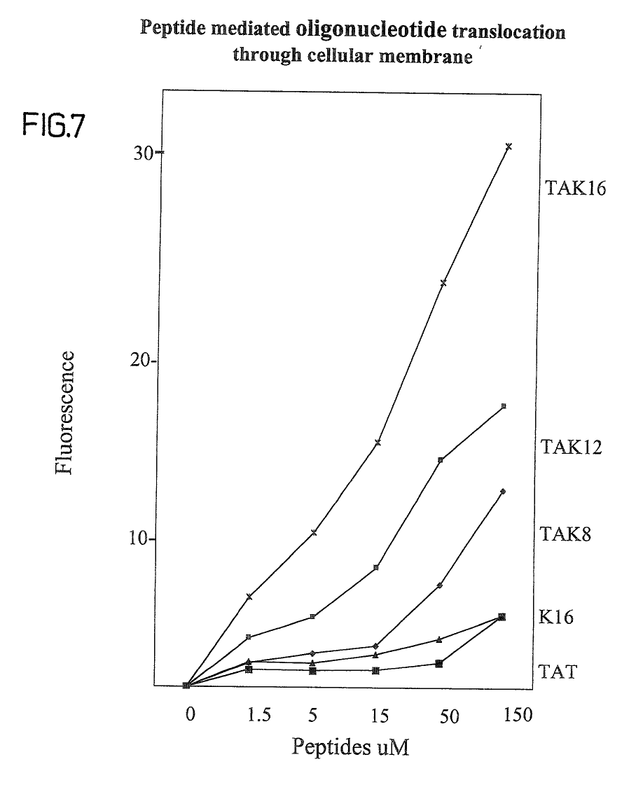

Next, various peptide sequences and domains were tested for their abilities to facilitate translocation of the oligonucleotide FITC-O-24 across a cell membrane. The peptides tested included: K16 (G₄K₁₆; SEQ ID NO: 47); TAT (G₄YGRKKRRQR₃; SEQ ID NO: 48); TAK8 (G₄YGRKKRRQR₃G₄K₈; SEQ ID NO: 49); TAK12 (G₄YGRKKRRQR₃G₄K₁₂; SEQ ID NO: 50) and TAK16/TatK₁₆ (G₄YGRKKRRQR₃G₄K₁₆; SEQ ID NO: 36). HeLa cells (1×10⁵) were incubated with the oligonucleotide (0.6 μl, 50 μM) in 10 μl DPBS and various concentrations of the peptides (i.e., 0.0, 1.5, 5.0, 15.0, 50.0, or 150 μM). The cells were incubated in the presence of the mixtures for fifteen minutes at room temperature and then analyzed by FACS. The results (FIG. 7) indicate that TAK16, which has the longest lysine adapter domain of the peptides used, was the most efficient in facilitating transport of FITC-O-24 (SEQ ID NO: 52) across the cell membrane. Neither TAT, nor adapter domain, K16, were able to efficiently facilitate transport. The data also reveal that, compared to TAK16, TAK12 and TAK8 had decreased ability to facilitate transport of an oligonucleotide, and the decreased ability was proportional to the decreased lengths of the lysine adapter domain.

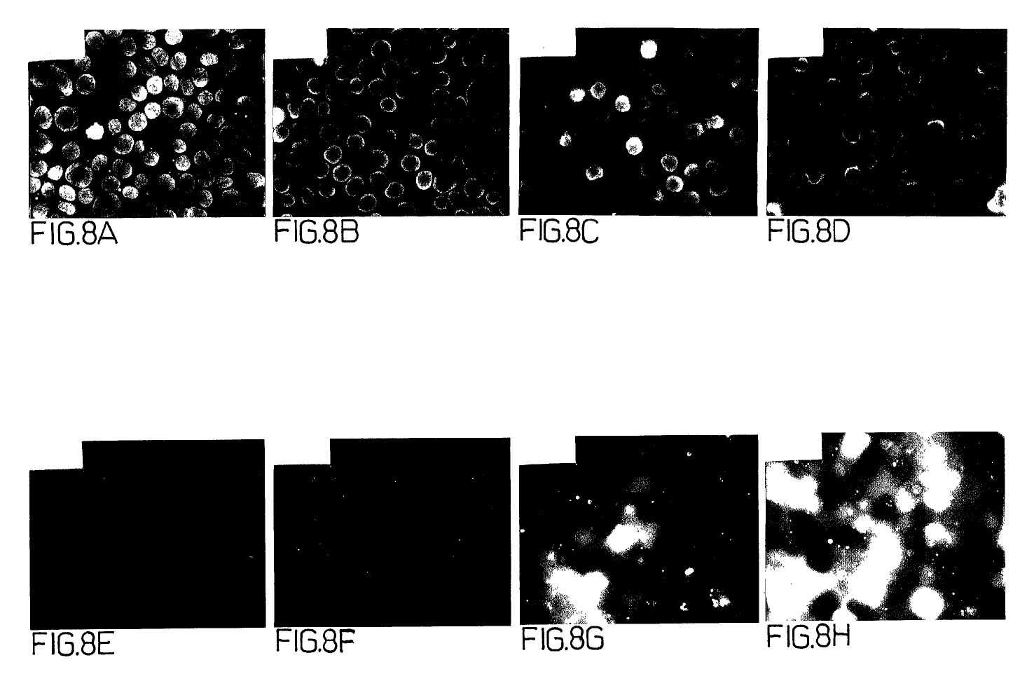

Next, the TAK16 peptide was further tested for its ability to facilitate uptake of the fluorescent labeled oligonucleotide FITC-O-24. HeLa cells were incubated with TAK16 at 0.00, 0.36, 1.80 or 9.00 μM and with FITC-O-24 (2.5 μM) as described above. The cells were prepared for fluorescence microscopy as described above by applying the cells to slides, counterstaining the DNA with DAPI for 5 minutes, covering the slides with antifade mounting medium (Vectastain), and then they were overlaid with a coverslip. It can be seen in the fluorescent micrographs of FIG. 8 that the fluorescent intensity associated with FITC-O-24 increased in the cells incubated with increased amounts of TAK16 peptide, indicating that TAK16, in a dose-dependent manner, can facilitate the uptake of a molecule such as an oligonucleotide.

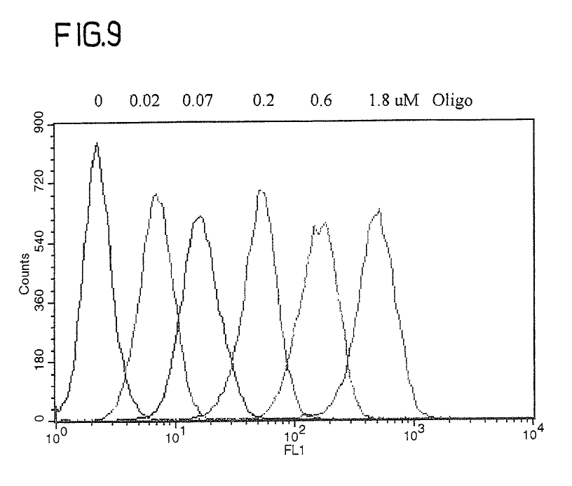

It was next determined whether TAK16 peptide could facilitate transport of an even larger oligonucleotide. HeLa cells were prepared as above and incubated with TAK16 at 13.3 μM and the fluorescein labeled oligonucleotide O-83-FITC (TTGATAAGAGGTCATTTTTGCGGATGGCTTAGAGCTTAATTGCTGAATCT GGTGCTGTAGCTCAACATGTTTTAAATATGCAA-3′-fluor; SEQ ID NO: 53) at concentrations of 0.00, 0.02, 0.07, 0.2, 0.6, or 1.8 μM. Cells were then subjected to FACS analysis or fluorescent microscopic analysis. The FACS analysis (FIG. 9) showed that at a fixed concentration of TAK16, the amount of cell associated fluorescent intensity increased with increasing amounts of the 83-mer to which the cells were exposed. The results also show that an oligonucleotide which has a fluorescent label on the 5′ end can be translocated.

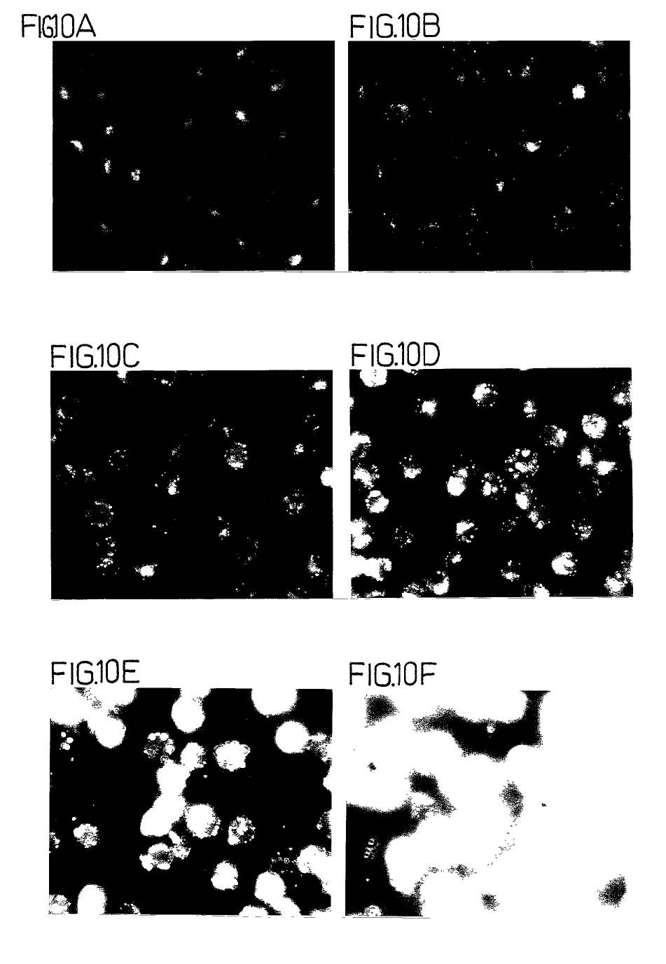

Some of the cells from the experiment depicted in FIG. 9, were also subjected to fluorescent microscopic analysis (FIG. 10). Results similar to those found above were obtained. The micrographs show that when cells were incubated with a fixed amount of TAK16 peptide, that when they were also subjected to increased amounts of fluorescein labeled oligonucleotide, there was also increased cellular associated fluorescence. This suggests that there were increased amounts of uptake of the oligonucleotide associated with the increased levels of the oligonucleotide in the incubation medium.

Peptide Mediated Translocation of DNA across Cell Membranes

Next, it was decided to determine whether even larger nucleotides, including double stranded (DS) DNA or plasmid DNA, could be transported into a cell. Specifically, it was determined whether TAK16 could facilitate the transport of 400 base pair (bp) double stranded DNA and a 5 kb circular plasmid DNA (pECFP; enhanced cyano fluorescent protein) into cells. The DNAs were labeled with Cy3 reagent (“Label IT” Cy3 Nucleic Acid Labeling Kit; Mirus, Madison, Wis.), according to manufacturer's directions. This protocol usually yields one label per up to ten nucleotides. To this end, HeLa cells were prepared as above and then incubated with 400 ng of plasmid DNA or 60 ng of 400 bp DNA and with TAK16 (12 μM).

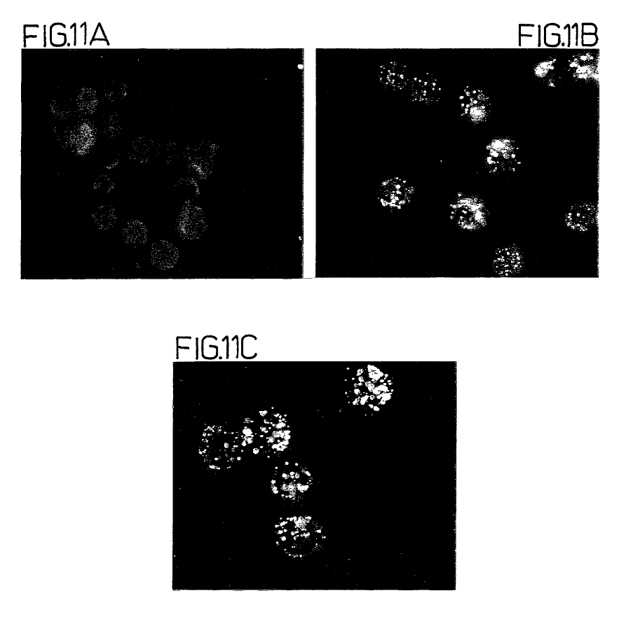

The results of this set of experiments are presented in the fluorescence micrographs of FIG. 11. The results indicate that plasmid DNA alone does not enter the cell in the absence of TAK16 (FIG. 11A). However, when the cells were incubated with 400 bp DS DNA or plasmid DNA in the presence of TAK16 peptide, there was obvious fluorescence. These data indicate that fusion peptides with adapter domains such as TAK16, facilitate transport of large DNA molecules into cells, and uptake of large double stranded DNA molecules, both linear and circular, occurs in virtually 100% of the cells.

Next, efficiency of plasmid DNA uptake was determined, as well as whether the plasmid DNA delivered into the cells was biologically active. SK-BR-3 cells were incubated with pECFP (Cy3 labeled) and TAK16 as described above. Controls included pECFP alone or pECFP plus lipofectin (positive control). Plasmid uptake was determined by measuring the plasmid marker, Enhanced Cyano Fluorescent Protein (ECFP). Following a 15 minute incubation, it was found that the translocation efficiencies were <1×10⁻² for plasmid DNA alone and 9.8×10⁻¹ for plasmid DNA and TAK16. A 15 minute incubation was not tested for lipofectin plus plasmid DNA. Forty-eight hours after addition of the various combinations, it was found that the translocation efficiencies were <1×10⁻² for plasmid DNA alone, 1.1×10⁻¹ for plasmid DNA and TAK16, and 2.9×10⁻¹ for plasmid DNA and lipofectin. The groups were also sampled two weeks later to determine the stability of transfection. It was found that at two weeks following exposure, the translocation efficiencies were <1×10⁻⁶ for plasmid DNA alone, 3×10⁻⁵ for plasmid DNA and TAK16, and 1×10⁻⁴ for plasmid DNA and lipofectin. The data demonstrate that, as compared to the lipofectin positive control, TAK16 is able to efficiently translocate plasmid DNA across SK-BR-3 cell membranes. The data further demonstrate that plasmid DNA translocated across the cell membrane by TAK16 remains biologically active, as measured by transient, 48 hour, and permanent expression assays.

The ability of TAK16 to facilitate translocation of oligonucleotides designed to form triplex DNA complexes with the SupFG1 sequence was tested in a murine cell line in which the SupFG1 gene is incorporated into the genome. The assay used measures chromosomal mutations induced in the SupFG1 gene by triplex forming oligonucleotides, as measured by mutations/total plaques. The ability of TAK16 to translocate triplex forming oligonucleotides was compared to that of Antennapedia (ANTP)-s-s-oligo complex and the lipid delivery system Gene Porter. The two oligonucleotides tested were AG13 (5′-AGGAA8-propylamine-3′; SEQ ID NO: 58) and AG30 (5′-AGGAAG8TGGTG5AG5AG-propylamine-3′; SEQ ID NO: 59). The groups tested were 1) control (no treatment); 2) AG13 alone; 3) AG13+Gene Porter; 4) AG13-s-s-ANTP; 5) AG13+TAK16; 6) AG30+Gene Porter; and 7) AG30+TAK16. The chromosomal mutation frequencies (×10⁻⁵) induced in the SupFG1 gene were found to be 7, 8, 9, 25, 40, 21 and 36, for groups 1-7, respectively. It can be seen that the two highest mutation rates occurred in cells treated with AG13+TAK16 (group 5; 40×10⁻⁵) and AG30+TAK16 (group 7; 36×10⁻⁵). Thus, the data demonstrate that not only can triplex oligonucleotides delivered by TAK16 induce mutations in a targeted gene in the chromosome of a living cell, that TAK16 was more efficient at inactivating the SupFG1 gene than either ANTP or Gene Porter.

In addition to the data disclosed herein for human and murine cells, the invention also works in frog (xenopus), zebra fish and C. elegans cell lines. Thus, these data of the present invention show that the invention works in eukaryotic cells, and suggest that it should work in prokaryotic cells as well.

Peptide-Oligo Mediated Uptake of Molecules

It was next determined whether a fusion peptide in combination with an oligonucleotide, could facilitate transport of a molecule across a cell membrane. In particular, it was determined whether TAK16 could facilitate transport of a combination of the oligonucleotide Bio-O-22 and a protein across a cell membrane. The protein chosen was FITC labeled avidin.

HeLa cells were prepared as above. Avidin-FITC in DPBS was mixed with Bio-O-22 (biotin-CCAGACTGAGTATCTCCTATCA; SEQ ID NO: 54) in DPBS to final concentrations of 12 μM and 8 μM, respectively. Then, 1.5 μl of the mix was added to 2×10⁵ cells in 10 μl DPBS. Next, 11.5 μl of 100 mM TAK16 was added, the cells were incubated for 20 minutes at 37° C., washed in DPBS, and subjected to fluorescent microscopy.

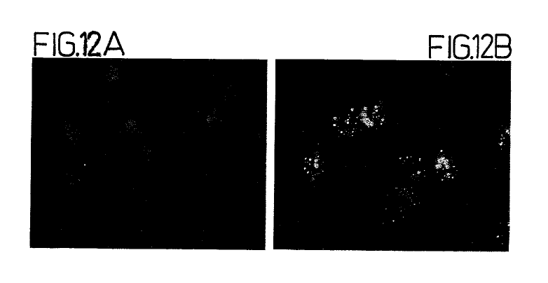

The data demonstrate that a fusion peptide can facilitate transport of an oligonucleotide (Bio-O-22) and a 67 kD peptide (avidin) across a cell membrane. It can be seen in FIG. 12 that the group which was incubated in the presence of TAK16 and avidin, but no oligonucleotide, did not exhibit appreciable levels of fluorescence (FIG. 12A). However, the group in which cells were incubated in TAK16 along with a mixture of the oligonucleotide and avidin, exhibited much higher levels of fluorescence (FIG. 12B) than in the cells which were not exposed to all three components (FIG. 12A).

To further determine the ability of a fusion peptide to facilitate transport of a molecule associated with a nucleic acid across a cell membrane, TAK16 was tested for its ability to mediate transport of another protein. In this example, the protein horseradish peroxidase (HRP) was tested. The HRP was conjugated to avidin to yield 120 kD Horseradish Peroxidase-Avidin (HPA). Cells were obtained as above. HPA and Bio-O-22 were added to cells in DPBS at final concentrations of 8 μM and 8 μM, respectively, and then incubated for 15 minutes at room temperature. Then, 2.5 μl of the mix was added to 2×10⁵ HeLa cells in 10 μl DPBS. Next, 12.5 μl of 100 μM TAK16 in DPBS was added to the mixture and incubated for 30 minutes at 37° C. Then the cells were washed and applied to slides. Cells on the slides were treated with 30 μl peroxidase suppressor solution to suppress endogenous peroxidase, washed with DPBS, treated with 30 μl Metal Enhanced DAB Substrate Working Solution, incubated for 15 minutes at room temperature, and then subjected to microscopic analysis. Positives were revealed utilizing a DAB kit and peroxidase suppressor (Pierce Chemical Co., Rockford, Ill.). HPA activity or presence was detected using the DAB kit and the manufacturer's protocol.

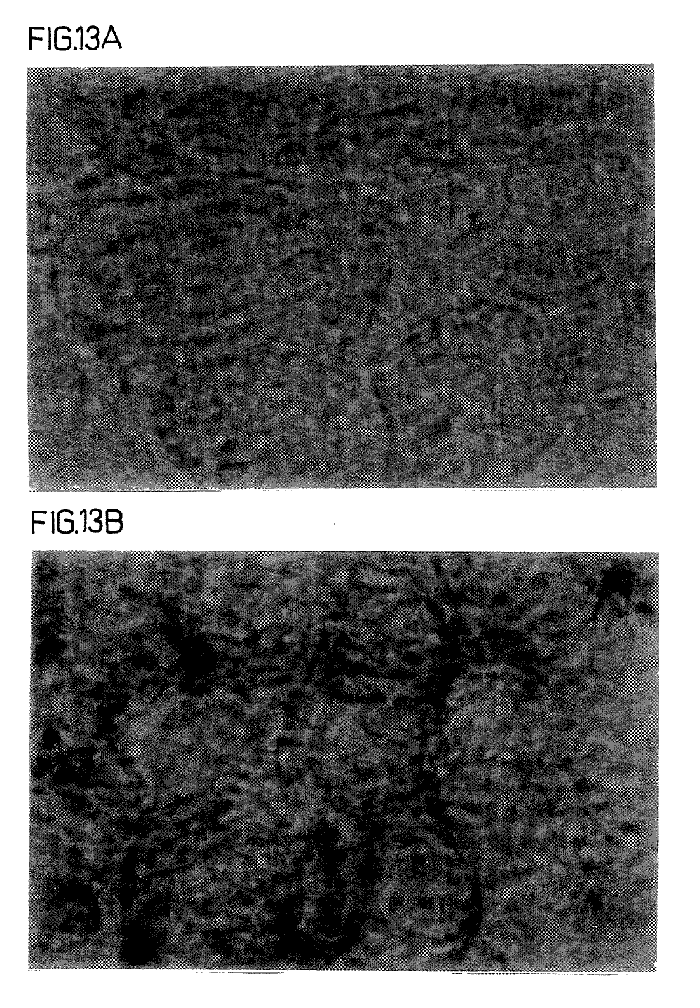

Histochemical analysis of the cells showed that HPA uptake was facilitated in the group exposed to HPA, TAK16, and the oligonucleotide (FIG. 13B), but not in the group exposed to only HPA and TAK16 (FIG. 13A).

In similar experiments, TAK16 peptide was also able to facilitate intracellular transport of a 140 kD protein (beta-galactosidase) conjugated to an oligonucleotide.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A fusion peptide comprising at least one cell penetrating domain which is linked to at least one transmembrane domain, wherein the orientation of the cell penetrating domain is independent of the orientation of the transmembrane domain.

2. An isolated nucleic acid encoding the fusion peptide of claim 1.

3. The fusion peptide of claim 1 wherein the cell penetrating domain is selected from the group consisting of SEQ ID NOs: 1-20 and 21.

4. An isolated nucleic acid encoding the fusion peptide of claim 3.

5. The fusion peptide of claim 1 wherein the transmembrane domain is selected from the group consisting of SEQ ID NOs: 22-27 and 45.

6. An isolated nucleic acid encoding the fusion peptide of claim 5.

7. The fusion peptide of claim 1, further comprising at least one ligand binding domain, wherein the orientation of the ligand binding domain is independent of the orientation of the cell penetrating domain and the transmembrane domain.

8. An isolated nucleic acid encoding the fusion peptide of claim 7.

9. The fusion peptide of claim 7, wherein the ligand binding domain comprises biotin.

10. The fusion peptide of claim 9, wherein the ligand binding domain comprises K8-biotin (SEQ ID NO: 34).

11. The fusion peptide of claim 7, wherein the ligand binding domain comprises a receptor for a polypeptide.

12. The fusion peptide of claim 11, wherein the polypeptide is selected from the group consisting of a growth factor, a cytokine, and a hormone.

13. The fusion peptide of claim 7, wherein the ligand binding domain comprises WDHFECSCTGLPF (SEQ ID NO: 46).

14. A fusion peptide comprising Composite Receptor 1 (CR1).

15. An isolated nucleic acid encoding the fusion peptide of claim 14.

16. A fusion peptide comprising Composite Receptor 2 (CR2).

17. An isolated nucleic acid encoding the fusion peptide of claim 16.

18. A fusion peptide comprising Composite Receptor 1R (CR1R).

19. An isolated nucleic acid encoding the fusion peptide of claim 18.

20. The fusion peptide of claim 7, wherein the ligand binding domain comprises an effector domain.

21. An isolated nucleic acid encoding the fusion peptide of claim 20.

22. An isolated peptide selected from the group consisting of:
(a) an isolated peptide comprising an amino acid sequence selected from the group consisting of SEQ ID NOs: 36, 38 and 39;
(b) an isolated peptide comprising a fragment of at least three amino acids of an amino acid sequence selected from the group consisting of SEQ ID NOs: 36, 38 and 39;
(c) an isolated peptide comprising conservative amino acid substitutions of the amino acid sequences selected from the group consisting of SEQ ID NOs: 36, 38 and 39; and
(d) naturally occurring amino acid sequence variants of amino acid sequences selected from the group consisting of SEQ ID NOs: 36, 38 and 39.

23. An isolated nucleic acid encoding the peptide of claim 22.

24. A composition comprising the fusion peptide of any one of claims 1, 3, 5, 7, 9-14, 16, 18, and 20, or the isolated peptide of claim 22, and a pharmaceutically acceptable carrier.

25. An isolated peptide selected from the group consisting of the amino acid sequence of SEQ ID NOs: 36, 38 and 39.

26. An isolated nucleic acid encoding the peptide of claim 25.

27. A method of incorporating a composite receptor into a cell membrane comprising administering to the cell an effective amount of a fusion peptide selected from claims 1, 3, 5, 7, 9-14, 16, 18, and 20, whereby the composite receptor incorporates into the cell membrane.

28. The method of claim 27, wherein the cell is a eukaryotic cell.

29. The method of claim 28, wherein the eukaryotic cell is a mammalian cell.

30. The method of claim 29, wherein the mammalian cell is a human cell.

31. A method of identifying a binding partner of a receptor domain of a composite receptor fusion peptide, the method comprising;
a. contacting a cell comprising the receptor domain with a test compound;
b. comparing the level of binding of the test compound with the cell with the level of binding of the test compound in an otherwise identical cell not comprising the receptor domain, wherein a higher level of binding of the test compound in the cell contacted with the test compound, compared with the level of binding of the test compound in the otherwise identical cell not comprising the receptor domain is an indication that the test compound is a binding partner of the receptor domain, thereby identifying a binding partner of a receptor domain of a composite receptor fusion peptide.

32. A binding partner of a receptor domain identified by the method of claim 31.

33. A kit for administering a composite receptor fusion peptide to a cell, the kit comprising a composite receptor fusion peptide, an applicator, and an instructional material for the use thereof.

34. The kit of claim 33, wherein the fusion peptide is selected from the group consisting of Composite Receptor 1, Composite Receptor 1R and Composite Receptor 2.

35. The kit of claim 33, wherein the cell is a eukaryotic cell.

36. The kit of claim 35, wherein the eukaryotic cell is a mammalian cell.

37. The kit of claim 36, wherein the mammalian cell is a human cell.

38. A fusion peptide comprising at least one cell penetrating domain linked to an adapter domain capable of binding to a molecule, wherein the orientation of the cell penetrating domain is independent of the orientation of the adapter domain.

39. The fusion peptide of claim 38, wherein the fusion peptide is capable of facilitating translocation of the molecule across a cell membrane.

40. An isolated nucleic acid encoding the fusion peptide of claim 38.

41. The fusion peptide of claim 38, wherein the molecule is a nucleic acid.

42. The fusion peptide of claim 38, wherein the cell penetrating domain is selected from the group consisting of SEQ ID NOs: 1-20 and 21.

43. An isolated nucleic acid encoding the peptide of claim 42.

44. The fusion peptide of claim 41 wherein the adapter domain is selected from the group consisting of SEQ ID NOs: 28-35, 47, 55, and 56.

45. An isolated nucleic acid encoding the peptide of claim 44.

46. The fusion peptide of claim 41 wherein the adapter domain is a chemical moiety selected from the group of consisting of (2-dimethylamino)ethyl methacrylate, polyallylamine, hydroxyapatite, polyethyleneimine, protamine, glucaramide polymers, polyamines, and N-substituted glycine (NSG) peptoids.

47. The fusion peptide of claim 41 wherein the nucleic acid is selected from the group consisting of an oligonucleotide, DNA, a ribozyme and RNA.

48. The fusion peptide of claim 38, wherein the cell is a eukaryotic cell.

49. The fusion peptide of claim 48, wherein the eukaryotic cell is a mammalian cell.

50. The fusion peptide of claim 49, wherein the mammalian cell is a human cell.

51. The fusion peptide of claim 38, wherein the fusion peptide is selected from the group consisting of SEQ ID NOs: 36, 41, 49, and 50.

52. An isolated peptide selected from the group consisting of:
(a) an isolated peptide comprising an amino acid sequence selected from the group consisting of SEQ ID NOs: 41, 49 and 50;
(b) an isolated peptide comprising a fragment of at least three amino acids of an amino acid sequence selected from the group consisting of SEQ ID NOs: 41, 49 and 50;
(c) an isolated peptide comprising conservative amino acid substitutions of the amino acid sequences selected from the group consisting of SEQ ID NOs: 41, 49 and 50; and
(d) naturally occurring amino acid sequence variants of amino acid sequences selected from the group consisting of SEQ ID NOs: 41, 49 and 50.

53. A composition comprising the fusion peptide of any one of claims 41, 42, 43, 46-51, or the isolated peptide of claim 52, and a pharmaceutically acceptable carrier.

54. An isolated peptide selected from the group consisting of the amino acid sequence of SEQ ID NOs: 41, 49 and 50.

55. A method of facilitating translocation of a molecule into a cell, the method comprising administering to the cell the molecule and an effective amount of a fusion peptide, wherein the fusion peptide comprises at least one cell penetrating domain linked to an adapter domain capable of binding to the molecule, further wherein the fusion peptide facilitates translocation of the molecule into the cell, thereby facilitating translocation of the molecule into the cell.

56. A method of facilitating translocation a nucleic acid into a cell, the method comprising administering to the cell the nucleic acid and an effective amount of a fusion peptide, wherein the fusion peptide comprises at least one cell penetrating domain linked to an adapter domain capable of binding to the nucleic acid, further wherein the fusion peptide facilitates translocation of the nucleic acid into the cell, thereby facilitating translocation of the nucleic acid into the cell.

57. The method of claim 55 or 56, wherein the cell is a eukaryotic cell.

58. The method of claim 57, wherein the eukaryotic cell is a mammalian cell.

59. The method of claim 58, wherein the mammalian cell is a human cell.

60. A method of identifying a fusion peptide comprising an adapter domain capable of translocating a molecule into a cell, the method comprising:
a. contacting a cell with a fusion peptide comprising a test adapter domain and a molecule;
b. comparing the level of translocation of the molecule into the cell with the level of translocation into an otherwise identical cell contacted with the molecule, wherein a higher level of translocation of the molecule into the cell is an indication that the fusion peptide comprising the test adapter domain is capable of translocating the molecule into the cell, thereby identifying a fusion peptide comprising an adapter domain capable of translocating a molecule into a cell.

61. The method of claim 60, wherein the molecule is a nucleic acid.

62. The method of claim 61, wherein the nucleic acid is selected from the group consisting of the nucleic acid sequence of SEQ ID NOs: 40, 52, 53, and 54.

63. The method of claim 61, wherein the nucleic acid is the oligonucleotide comprising SEQ ID NO: 40.

64. An adapter domain identified by the method of claim 61.

65. The method of claim 60, wherein the cell is a eukaryotic cell.

66. The method of claim 65, wherein the eukaryotic cell is a mammalian cell.

67. The method of claim 66, wherein the mammalian cell is a human cell.

68. A kit for administering the fusion peptide of claim 41 to a cell, the kit comprising the fusion peptide, an applicator, and an instructional material for the use thereof.

69. A kit for administering the fusion peptide of claim 41 to a cell, wherein a molecule is bound to the adapter domain, the kit comprising the fusion peptide, a molecule, an applicator, and an instructional material for the use thereof.

70. The kit of claim 69, wherein the molecule is a nucleic acid.

71. A method of facilitating translocation of a molecule across a cell membrane, the method comprising administering the molecule and an effective amount of a fusion peptide and a nucleic acid to the cell, wherein the fusion peptide and nucleic acid facilitate translocation of the molecule across the cell membrane, thereby translocating the molecule across the cell membrane.
